(19) United States
(12) Patent Application Publication
(10) Pub. No.: US 2023/0153539 A1
(43) Pub. Date: May 18, 2023

(54) MACHINE-ASSISTED MEDICAL PATIENT INTERACTION, DIAGNOSIS, AND TREATMENT

(71) Applicant: Decoded Health, Inc., Berkeley, CA (US)

(72) Inventors: Brecken Uhl, Placitas, NM (US); Kevin Bayes, West Harbour (NZ); Shimi Baliti, Kumeu (NZ); Mark Hanson, Orinda, CA (US); Brent Sugimoto, Richmond, CA (US)

(21) Appl. No.: 17/277,001

(22) PCT Filed: Mar. 16, 2021
(86) PCT No.: PCT/US2021/022519

§ 371 Date: Mar. 17, 2021

Related U.S. Application Data
(60) Provisional application No. 62/990,829, filed on Mar. 17, 2020.

(51) Int. Cl. G06F 40/35; G16H 10/60; G16H 10/20; G06N 3/084; G06F 40/166; G06F 3/0484; G06F 3/0482

(52) U.S. Cl. CPC G06F 40/35; G06F 3/0482; G06F 3/0484; G06F 40/166; G06N 3/084; G16H 10/20; G16H 10/60; G06F 40/284

(57) ABSTRACT

A data-holistic optimized patient-physician technology (DH-OPPT) system, embodied in a set of one or more modular subsystems, may provide a unique approach to the clinical medical process to achieve enhanced efficiency and accuracy at many points throughout the end to end process of providing clinical medical care. Among other actions, the system may access conversation data that includes pairs of text passages from a dialog with a patient and then input the conversation data into a machine learning model trained to perform inference of medical conditions based on pairs of text passages. The trained machine learning model may output an inferred medical condition of the patient. The system may cause revision of an electronic health record of the patient based on the inferred medical condition.

### RELATED APPLICATION

This application claims the priority benefit of U.S. Provisional Pat. Application No. 62/990,829, titled “A SYSTEM AND METHOD FOR PERFORMING MACHINE-ASSISTED MEDICAL PATIENT INTERACTION, DIAGNOSIS, AND TREATMENT” and filed Mar. 17, 2020, which is incorporated herein by reference in its entirety.

### TECHNICAL FIELD

The subject matter disclosed herein generally relates to the technical field of special-purpose machines that facilitate interaction, diagnosis, or treatment for a medical patient, including software-configured computerized variants of such special-purpose machines and improvements to such variants, and to the technologies by which such special-purpose machines become improved compared to other special-purpose machines that also facilitate interaction, diagnosis, or treatment for a medical patient.

### BACKGROUND

The practice of modern medicine is highly dependent on healthcare information processing systems (HIPS). Healthcare workers, such as nurses, physicians, scribes, and technicians may interact with one or more HIPS to acquire, process, store, transport, and display patient information, as well as derivative work products and documentation based on that information. HIPS presently constitute a critical element in the workflow for delivering healthcare. Unfortunately, current HIPS suffer from low efficiency and effectiveness, as well as fall short of providing holistic application of the data available in a system. These shortfalls result from poor design concepts and execution, a lack of user-centric design, a lack of integration, a lack of user-enhancing features and capabilities, and a lack of holistic application of the available data.

### BRIEF DESCRIPTION OF THE DRAWINGS

Some embodiments are illustrated by way of example and not limitation in the figures of the accompanying drawings.

FIG. 1 is a diagram showing a data-holistic optimized patient-physician technology (DH-OPPT) system, embodied in an example realization as eleven modular subsystems, according to some example embodiments.

FIG. 2 is a diagram showing an example realization of a Conversation and Session Management subsystem, according to some example embodiments.

FIG. 3 is a diagram showing an example progression of a DH-OPPT graph and slot-based conversation that builds up the Conversation Memory shown in FIG. 2, according to some example embodiments.

FIG. 4 is a diagram showing an example realization of screen presented by the Technician in the Loop subsystem during a patient interaction, according to some example embodiments.

FIG. 5 is a diagram showing an example realization of neural network models, in the Medical Inference subsystem, used to perform inference around a patient’s medical condition, according to some example embodiments.

FIG. 6 is a diagram showing an example realization of a physician-level user interface in the Physician Encounter Interface subsystem, according to some example embodiments.

FIG. 7 is a diagram showing an example session management interface presented by the Technician in the Loop subsystem and in which one or more active sessions are listed and can be accessed, according to some example embodiments.

FIG. 8 is a diagram showing an example realization of an interaction between the Technician in the Loop subsystem and one or more other subsystems of the DH-OPPT system, where the Technician in the Loop subsystem is identifying and selecting semantically-relevant tokenized data elements, supporting or supported by one or more other subsystems, such as the Medical Inference subsystem and the Conversation and Session Management subsystem, according to some example embodiments.

FIG. 9 is a diagram showing an example realization of an interface of the Technician in the Loop subsystem, where the Technician in the Loop subsystem is enabled to select from various automatically derived data elements, edit such data elements, and save (e.g., finalize or otherwise commit) such data elements, summaries thereof, or candidate questions, according to some example embodiments.

FIG. 10 is a diagram showing an example interface to a graph-based conversational element, realization, where the interface of the Technician in the Loop subsystem is used to inspect the patient’s conversation with the DH-OPPT system, alongside the tokenized, state-aware, graph memory that is driving the conversation, as optionally moderated by the Technician in the Loop subsystem, according to some example embodiments.

FIG. 11 is a diagram showing an example interface where the Technician in the Loop subsystem is able to review, query, modify, and approve an automatically-generated and fully source-linked clinical encounter summary (e.g., before the summary is accessed by the Physician Data Preparation and Dynamic Scheduler subsystem), according to some example embodiments.

FIG. 12 is a diagram showing an example realization of an interface that enables a physician-level user to interact with a summary of a clinical encounter, where the summary features fully-traceable tokenized data and drive data elements, according to some example embodiments.

FIG. 13 is a diagram showing an example of a touch-enabled interface that enables a physician-level user to perform one or more diagnostic activities, with one or more displayed data elements, one or more derived data elements (e.g., derived tokens and derived objects), or both, according to some example embodiments.

FIG. 14 is a block diagram showing an example of a software architecture for a computing device, according to some example embodiments.

FIG. 15 is a block diagram of a machine in the example form of a computer system, within which instructions may be executed for causing the machine to perform any one or more of the methodologies discussed herein, according to some example embodiments.

FIG. 16 is a diagram showing an example of training and use of a machine learning program that may be used to deploy various example embodiments of any one or more of the systems and methodologies discussed herein.

FIG. 17 is a flowchart showing a method of operating a DH-OPPT system, according to some example embodiments.

### DETAILED DESCRIPTION

Example methods (e.g., algorithms) facilitate interaction, diagnosis, treatment, or any suitable combination thereof, for a medical patient, and example systems (e.g., special-purpose machines configured by special-purpose software) are configured to facilitate interaction, diagnosis, treatment, or any suitable combination thereof. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will be evident to one skilled in the art, however, that the present subject matter may be practiced without these specific details.

In one example of the use of a HIPS, the patient interactively enters information into a computer system.

In another example of the use of the HIPS, a technician reviews patient information in a computer system in order to schedule a patient encounter.

In another example of the use of the HIPS, a nurse or technician enters patient data into a computer system to record patient encounter information prior to the patient seeing a Physician.

In another example of the use of the HIPS, a Physician reviews patient data in the patient record.

In another example of the use of the HIPS, a Physician reviews reference material to support their diagnosis or prescriptive action.

In another example of the use of the HIPS, a Physician enters and manipulates data in a computer system in order to record a patient encounter.

In another example of the use of the HIPS, a Physician enters data in a computer system in order to produce a record of a prescription or referral for a patient.

In another example of the use of the HIPS, a technician schedules follow-up care with a patient.

In general, current HIPS without the methodologies discussed herein:
1) Seek to replicate manual workflows by adding technology elements on top of those workflows, often adding more time and cognitive burden than they remove.
2) Are not integrated, and have inconsistent data formats, interfaces, and workflows which are not time efficient, and place undue cognitive burden on the user.
3) Are not user-centric. Methods of interaction with the system are coarse and, though often information-dense, do little to organize the information in a readily-applied format; this issue extends to user modification of the information to produce derivative products.
4) Do not make holistic use of all of the available information, and do not enhance the effectiveness of the user. Often, the amount of data available to the healthcare provider is much more than a single user can observe and assess using current approaches.
5) Do not enhance user effectiveness or capabilities through the application of advanced processing rising to the level of what can be achieved by the users themselves.

One aspect of the state of present HIPS can be summed up with the phrase “death by a thousand clicks,” which intends to express severe frustration with many of the characteristics of current HIPS, as enumerated in points 1-5 above. The mere ingestion, display, processing, and manipulation of data into a digital format does not, in and of itself, provide significant value to the actual healthcare worker user. In fact, the quest for “digitization” for its own sake places even more burden on the user and makes the provision of healthcare even less efficient, especially for the absolutely most scarce and expensive resource: the physician-level user.

In contrast, example embodiments of the systems and methods described herein apply integrated, user-centric automation that is enabled by machine learning techniques to realize modular, but comprehensive, systems and methods for achieving otherwise unattainable efficiencies in medical patient interaction, diagnosis, treatment, or any suitable combination thereof, where the effectiveness of the medical staff users is enhanced simultaneous to achieving such efficiency.

The domains of various example embodiments include the practice of medicine, information technology, cloud computing, application programming, and artificial intelligence. Those practiced in these arts will clearly recognize the novelty and utility of the example embodiments described herein, as well as understand that example realizations of the example embodiments do not constitute a limitation to what is described in the present subject matter.

Example embodiments may achieve one or more capabilities described herein, merely by way of example, for a DH-OPPT system. One or more of such example embodiments may address several critical and unmet needs with regard to optimizing efficiency and accuracy for both patients and physicians engaged in medically-related activities and interactions. To achieve this, various example embodiments discussed herein realize a DH-OPPT system with a system (e.g., a computer system or a client-server system) of one or more machines that has the following example capabilities:
1. Streamlined patient interactions, for example telehealth and chat interfaces, voice interactions, information-optimized conversations, or any suitable combination thereof.
2. Integrated use of all available data, for example including clinical guidelines, reports, electronic health records (EHR), conversations, feedback, or any suitable combination thereof.
3. Automated use of all available data, for example including automated data processing, machine learning, artificial intelligence, data summarization, or any suitable combination thereof.
4. Maximized use of all available data, for example including machine learning, active learning, or any suitable combination thereof.
5. Streamlined physician interactions with the patient, the information processing system (e.g., the DH-OPPT system), or both, for example including data summarization, explainable decision support, automated note generation, or any suitable combination thereof.
6. Maximized physician precision, for example including medical condition inference, predictive actions customized to the patient, predictive outcomes, or any suitable combination thereof.
7. Automated interaction events and data resolution, for example including dynamic interaction, scheduling, automated follow-up, data injection, billing system interface, or any suitable combination thereof.

The benefits of Streamlined Patient Interactions (1) may include, but are not limited to: improved access, improved data gathering from the patient, improved efficiency, or any suitable combination thereof. Furthermore, this streamlined interaction may improve the overall efficiency and accuracy of medical care, which can lower the cost of care (e.g., cost efficiency), paid for directly or through some third-party provider such as an employer, insurer, or government benefit.

The benefits of Integrated Use of All Available Data (2) may include, but are not limited to: improved physician situational awareness, improved diagnostic precision, improved patient outcomes, reduction in liability risk, or any suitable combination thereof.

The benefits of Automated Use of All Available Data (3) may include, but are not limited to specialization of diagnosis and medical care actions for the individual patient based on their record (e.g., as well as global patient cohort records), maximization of data analysis efficiency, maximization of physician information assessment efficiency, maximization of completeness, efficiency of documentation, or any suitable combination thereof.

The benefits of Maximized Use of All Available Data (4) may include, but are not limited to: optimization of diagnosis and medical care actions, optimization of patient outcomes, optimization of available healthcare resources, discovery of new relationships within the data, or any suitable combination thereof.

The benefits of Streamlined Physician Interactions with the Patient and Information Processing System (5) include, but are not limited to: efficient use of physician time, improved diagnostic accuracy, improved data collection and documentation accuracy and clarity, thoroughness of follow-up care, or any suitable combination thereof.

The benefits of Maximized Physician Precision (6) may include, but are not limited to: improved efficiency of use of medical resources, improved patient outcomes, amplified improvement of all inference-driven capabilities through improvements to data quality, or any suitable combination thereof.

The benefits of Automated Interaction Events and Data Resolution (7) may include, but are not limited to: improved efficiency and quality of care through dynamic scheduling, improved efficiency and consistency of follow-up care, improved data quality by finalizing outcomes, or any suitable combination thereof.

The benefits just described for each DH-OPPT capability are not intended to be limiting. The benefits realized from the combination of all of the above-described capabilities may be even more beneficial when used in combination, which may realize a totally unprecedented ability to achieve efficient and precise medical patient interaction, diagnosis, treatment, or any suitable combination thereof.

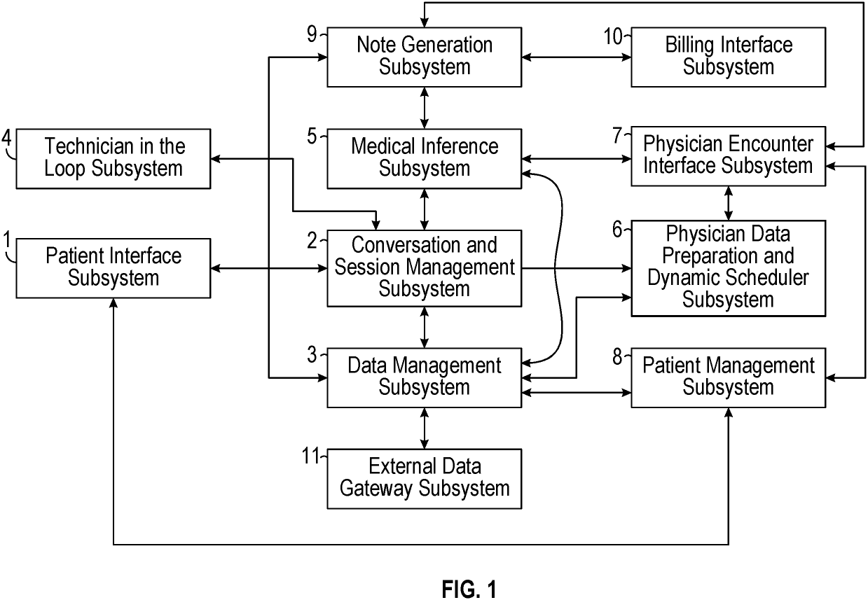

FIG. 1 is a diagram showing a DH-OPPT system, embodied in an example realization as eleven modular subsystems, according to some example embodiments. As shown in FIG. 1, the DH-OPPT system may include any one more of the following subsystems: a Patient Interface subsystem 1, a Conversation and Session Management subsystem 2, a Data Management subsystem 3, a Technician in the Loop subsystem 4, a Medical Inference subsystem 5, a Physician Data Preparation and Dynamic Scheduler subsystem 6, a Physician Encounter Interface subsystem 7, a Patient Management subsystem 8, a Note Generation subsystem 9, a Billing Interface subsystem 10, and an External Data Gateway subsystem 11.

The Patient Interface subsystem 1 interfaces (e.g., directly) with the Conversation and Session Management subsystem 2, the Patient Management subsystem 8, or both. The Patient Interface subsystem 1 performs patient-facing functions, such as enrollment, account management, medical assistance session initiation, medical assistance session conversation question and answer entry and display, scheduling selection and display, telehealth session interface, event notification, and access to account records.

The Conversation and Session Management subsystem 2 is an executive agent that coordinates between the Patient Interface subsystem 1, the Data Management subsystem 3, the Technician in the Loop subsystem 4, the Medical Inference subsystem 5, or any suitable combination thereof. The Conversation and Session Management subsystem 2 may use machine intelligence to drive a flexible, agenda-aware, and slot-oriented patient interaction, which may be under supervision by the Technician in the Loop subsystem 4. The Conversation and Session Management subsystem 2 accesses and stores data through the Data Management Subsystem 3, using such data to drive the patient conversation, a function which applies machine intelligence provided by the Medical Inference subsystem 5. Once the conversation has proceeded to an actionable endpoint, the Conversation and Session Management subsystem 2 transfers control to the Physician Data Preparation and Dynamic Scheduler subsystem 6.

The Data Management subsystem 3 interfaces to one or more of the other subsystems to provide data storage, access, and discovery services. Patient personally-identifiable information (PII) may be protected in the Data Management subsystem 3 through the use of strict access controls, minimum-access policies, the implementation architecture, encryption, or any suitable combination thereof.

The Technician in the Loop subsystem 4 provides an interface for information display, modification, and approval by a qualified medical technical user who may optionally supervise a patient conversation, take full control of a patient conversation, supervise an information summary, or any suitable combination thereof, prior to handoff of the encounter to a physician-level user. The Technician in the Loop subsystem 4 may be driven by the Conversation and Session Management subsystem 2, with data access provided to drive the primary patient conversation, as well as produce the final derivative conversation produced by the Physician Data Preparation and Dynamic Scheduler subsystem 6. The interface in the Technician in the Loop subsystem 4 affords efficient and accurate conversation management, information labeling, and information approval by the medical technician user, who is able to handle multiple federated tasks across multiple conversations simultaneously.

The Medical Inference subsystem 5 provides machine intelligence services to the rest of the DH-OPPT system and may interface (e.g., directly) to the Conversation and Session Management subsystem 2, the Physician Encounter Interface subsystem 7, the Note Generation subsystem 9, the Data Management subsystem 3, or any suitable combination thereof. The Medical Inference subsystem 5 obtains information from and provides information to one or more of the other subsystems (e.g., indirectly) through the Data Management subsystem 3. The Medical Inference subsystem 5 may be used to drive patient conversations, intelligently organize information, perform inference as to patient condition, perform inference as to recommended actions, perform inference as to expected outcomes, assist in note and record generation, aid in scheduling and follow-up, or any suitable combination thereof.

The Physician Data Preparation and Dynamic Scheduler subsystem 6 interfaces directly with the Conversation and Session Management subsystem 2, the Physician Encounter Interface subsystem 7, the Data Management subsystem 3, or any suitable combination thereof. The Physician Data Preparation and Dynamic Scheduler subsystem 6 acquires session control from the Conversation and Session Management subsystem 2, determines scheduling based on present data, availability of resources, patient and healthcare user input, or any suitable combination thereof, and also organizes data for subsequent presentation, later modification, derivative product generation, or any suitable combination thereof, by the physician-level user in the Physician Encounter Interface subsystem 7.

The Physician Encounter Interface subsystem 7 interfaces (e.g., directly) with the Medical Inference subsystem 5, the Physician Data Preparation and Dynamic Scheduler subsystem 6, the Patient Management subsystem 8, the Note Generation subsystem 9, or any suitable combination thereof. The data provided by the Physician Data Preparation and Dynamic Scheduler subsystem 6 is made available for display, modification, derivative product generation, or any suitable combination thereof, in the Physician Encounter Interface subsystem 7. The Medical Inference subsystem 5 may interact with the physician-level user as they display, manipulate, or generate information in the Physician Encounter Interface subsystem 7. The physician-level user may use the Physician Encounter Interface subsystem 7 to interact with the Patient Management subsystem 5 to implement one or more actions. The Physician Encounter Interface subsystem 7 and the physician- level user may interact with the Note Generation subsystem 9 to create one or more patient encounter notes or other records, including records relevant to the Billing Interface subsystem 10.

The Patient Management subsystem 8 interfaces (e.g., directly) with the Physician Encounter Interface subsystem 7, the Data Management subsystem 3, the Patient Interface subsystem 1, or any suitable combination thereof. The Patient Management subsystem 8 may provide direct and automated interaction cues and messaging between or among the DH-OPPT system, one or more system users, the patient, one or more third-party systems (e.g., systems of pharmacies, laboratories, or any other healthcare-related system, possibly except insurance billing, which may be handled by the Billing Interface subsystem 10).

The Note Generation subsystem 9 interacts (e.g., directly) with the Medical Inference subsystem 5, the Physician Encounter Interface subsystem 7, the Data Management subsystem 3, the Billing Interface subsystem 10, or any suitable combination thereof. Based on the physician-level user’s editing (e.g., with finalization) of the patient information or a derivative product, such as an encounter note, the Note Generation subsystem 9 may leverage the capabilities of the Medical Inference subsystem 5 to produce automated documentation and record entries, which may then be stored in the Data Management subsystem 9 and made available to the Billing Interface subsystem 10.

The Billing Interface subsystem 10 interacts (e.g., directly) with the Note Generation subsystem 9 and may interact (e.g., indirectly) with the Data Management subsystem 3. The Billing Interface subsystem 10 may provide an automated transfer of patient encounter information (e.g., to a third-party billing system, in a format suitable for the third-party billing system).

The External Data Gateway subsystem 11 provides a secure interface and data format translation to one or more external resources, such as third-party electronic health records (EHRs). The External Data Gateway subsystem 11 may be controlled by the Data Management subsystem 3.

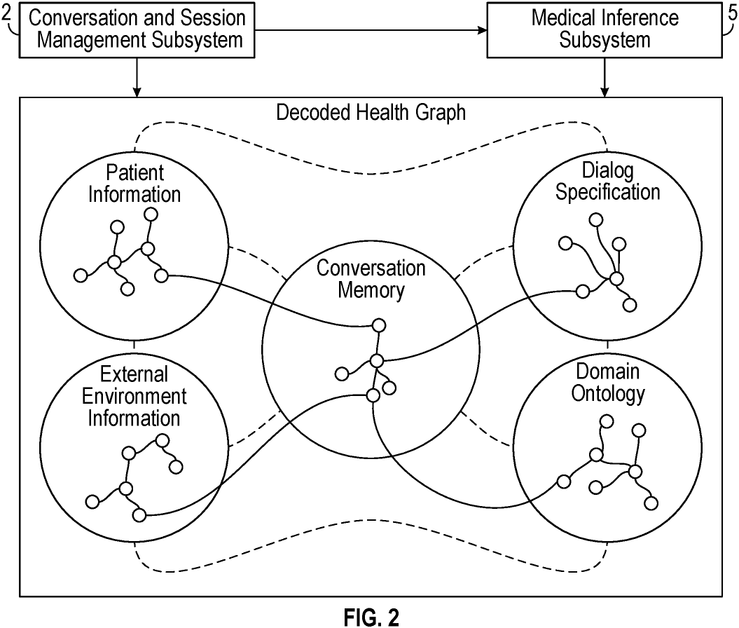

FIG. 2 is a diagram showing an example realization of the Conversation and Session Management subsystem 2, according to some example embodiments. The Conversation and Session Management subsystem 2 works with one or more of several other DH-OPPT subsystems to achieve a natural, efficient, and information-dense patient conversation experience. The Conversation and Session Management subsystem 2 drives the patient conversation with a slot-oriented, graph-based canonical dialog approach, enhanced by several artificial intelligence-driven services supplied by the Medical Inference subsystem 5. The Conversation and Session Management subsystem 2 integrates data from multiple sources, including one or more in-system or external electronic patient records, one or more context and intent sensitive dialog specifications, the patient conversation, one or more structured hierarchical semantic domain ontologies (e.g., SNOMED Clinical Terms (SNOMED-CT)), or any suitable combination thereof.

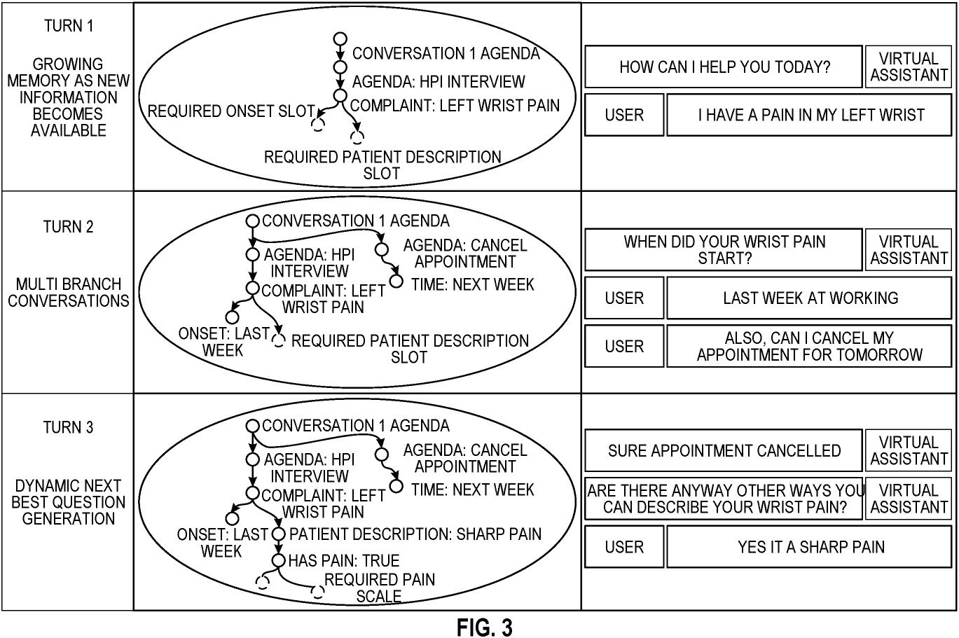

FIG. 3 is a diagram showing an example progression of a DH-OPPT graph and slot-based conversation that builds up the Conversation Memory, which is shown in FIG. 2, according to some example embodiments. This unique capability leverages the Medical Inference subsystem 5 to identify one or more medical contexts in the patient conversation, as well as dynamically create questions for the patient that lead to maximum information extraction with the fewest number of questions. The Medical Inference subsystem 5 and the Conversation and Session Management subsystem 2 may each draw information from the patient’s electronic medical records, which may enable a context-rich and personalized conversational experience. The graph and slot based approach enables a natural flow of the conversation, as well as context-switching with returns to one or more prior contexts or intents until all or a sufficient number of slots have been addressed. The extremely flexible and inference-driven approach of the example embodiments described herein starkly contrasts with non-holistic approaches that do not synthesize and utilize all available information in the way described herein, or that do not bring together the capabilities of a slot and graph-based conversation management approach dynamically throughout multiple contexts and intents.

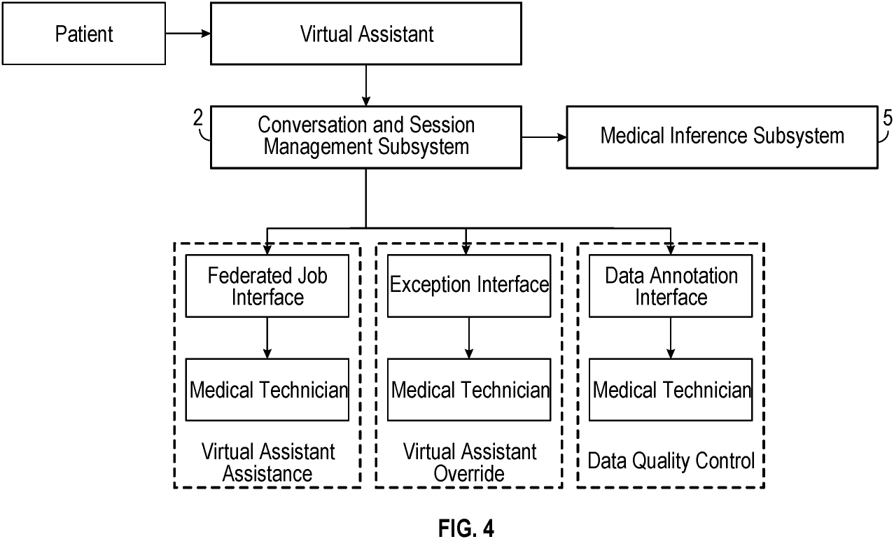

FIG. 4 is a diagram showing an example realization of a screen presented by the Technician in the Loop subsystem 4 during a patient interaction, according to some example embodiments. During the patient interaction, the medical technician is able to efficiently switch between and among multiple sessions, label one or more medical terms, flag one or more exceptions, or any suitable combination thereof.

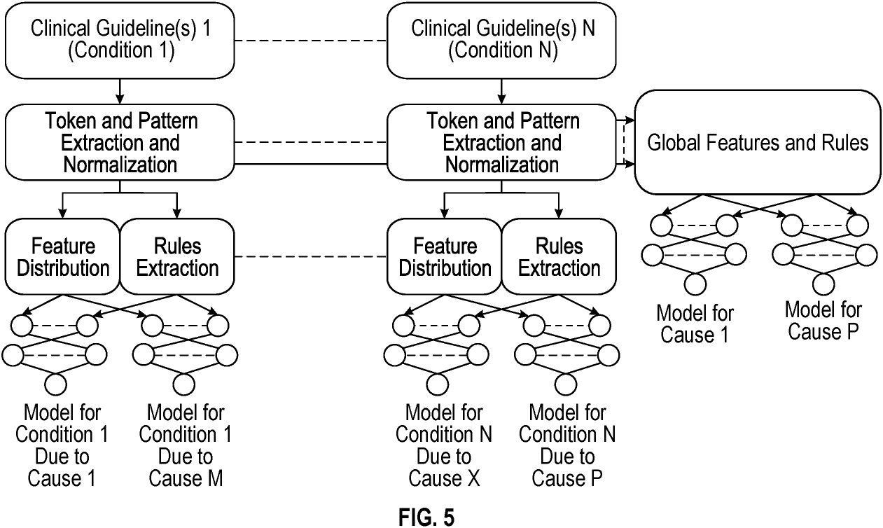

FIG. 5 is a diagram showing an example realization of neural network models, in the Medical Inference subsystem 5, used to perform inference around one or more patient medical conditions, according to some example embodiments. The neural network shown provides data normalization of patient conversation data and patient record data to a standardized information space (e.g., an ontology, such as SNOMED-CT). Inference may be achieved by computing conditions per cause and individual cause probabilities, resulting in a composite probability metric for each condition-cause pairing. One or more of the models may be built from nodes that include computable rules extracted from free-text medical guidelines, nodes consisting of individual raw data features, or any suitable combination thereof. The architecture of the neural network may use any classical or modern approach generally used in current best-practices, such as a multi-layer deep neural network with a fully-connected output layer, a recurrent neural network, a transformer-based network, or any suitable combination thereof or variation thereof.

However, the herein described use of extracted rules, the herein described initial weightings of broad term-oriented distributional features, and suitable combinations thereof, may provide a unique “cold-start” capability to the example embodiments described herein, which may be embedded in a contemporary architecture that can be improved by using an equivalent of a gradient backpropagation class of technique. Furthermore, the aspect of “explainability,” including explainability based on cause and effect, as described below, may provide one or more benefits that include human interaction with the models, trust of the models, and the ability to discover and enumerate new findings revealed in the data and in the models, as the models are trained over time.

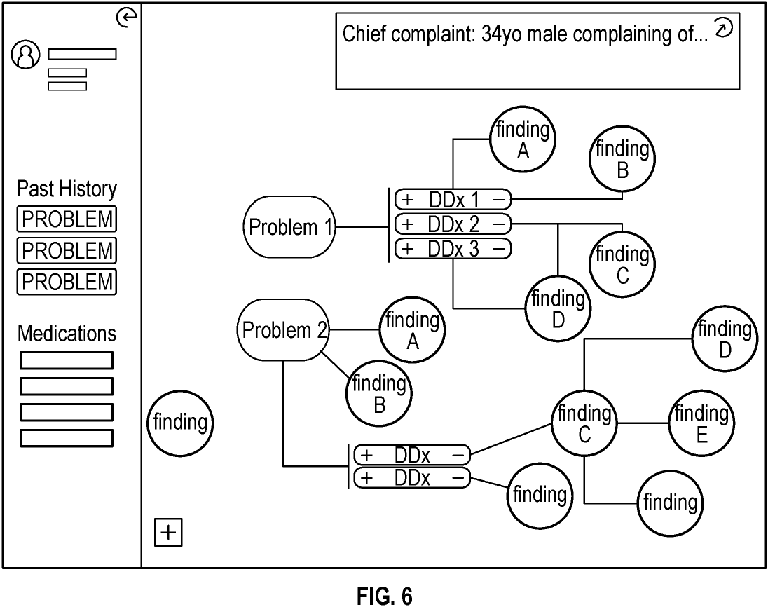

FIG. 6 is a diagram showing an example realization of a physician-level user interface (e.g., a graphical user interface (GUI)) in the Physician Encounter Interface subsystem 7, according to some example embodiments. In the example realization shown, the rich data format, as optionally moderated, formatted, edited (e.g., revised), and saved (e.g., finalized or otherwise committed) by the Technician in the Loop subsystem 4, is supplied by the Physician Data Preparation and Dynamic Scheduler subsystem 6, which may initialize a display as shown in FIG. 6. In the display, interface elements include: basic information (e.g., patient account, encounter, demographics, or any suitable combination thereof); past medical history (e.g., extracted from the DH-OPPT system, a third party EHR, one or more other relevant resources, or any suitable combination thereof); patient-reported medications (e.g., not shown in the past medical history), a textual summary of the encounter, based on present findings (e.g., as seen in the upper right portion of FIG. 6); and a graph-based display relating medical problems, associated inferred differential diagnosis possibilities, findings associated with those differential diagnosis possibilities, or any suitable combination thereof.

Using the interface shown in FIG. 6, the physician-level user is able to dive deeper into any of the displayed data elements, such as by accessing more explicit record information about the patient’s EHR data, the source of one or more findings, a list of findings important to a displayed differential diagnosis (e.g., which may not yet have been found in the encounter data), one or more reference resources relating to the displayed differential diagnosis list, or any suitable combination thereof. The interface may enable the physician-level user to add or delete problems, differential diagnoses, findings, or any suitable combination thereof, rearrange the data that is shown, and edit each element’s association with one or more other elements.

Once any editing is complete, the physician-level user saves (e.g., finalizes or otherwise commits to storage) the findings, which may have been automatically pre-formatted by the Note Generation subsystem 9 (e.g., in conjunction with the Medical Inference subsystem 5. Next, the physician-level user may proceed to establish one or more actions or expected outcomes in a similar interface, which may include advanced automation and pre-population capabilities as described herein, after which the physician-level user then may move into the patient encounter, note generation, or other derivative data product generation portions (e.g., phases) of the workflow. This multi-tiered format, expressed in a graphical and easily manipulated interface, and pre-populated and post-supported by machine learning medical inferences based on a holistic expression of all patient data in the context of a wider medical cohort, is far beyond the state of the art and provides many benefits, including improved efficiency, improved accuracy, improved basis of support, and reduced cognitive load benefits.

By virtue of the systems and methods described herein, an end-end streamlined process for medical patient interaction, diagnosis, and treatment is possible to implement in a new type of HIPS. Conventional HIPS focus on a human-intensive data interaction, assessment, and documentation model. Previous attempts at automation focused on only a single element of the process, as well as added data-entry burdens to the healthcare delivery workflow. In contrast, the various example embodiments discussed herein reduced the amount of manual interaction by both patients and medical users, thus achieving time and cognitive load reductions, simultaneously with improving the effectiveness of the medical care provided.

### 1 - Example Patient Interface Subsystem

The Patient Interface subsystem 1, according to various example embodiments, performs some or all of the functions to interface the patient to the automated portions of the DH-OPPT system. The patient may initiate an interaction event with the DH-OPPT system, using means such as telephony, a computer interface for registration and patient data management, a computer interface for text chat, a computer interface for voice and text chat, a computer interface for text and video chat, or any suitable combination thereof. These interaction events afford the patient the ability to interact with the DH-OPPT system using a mode that is best matched to their needs or that they find most convenient. The information gathered from the patient in the interaction event will be used later in the care of the patient, which differentiates the interaction event from a condition-checker or a scheduler in capability. Furthermore, the variety and seamlessness of interaction modes with the patient maximizes utilization of the DH-OPPT system. The flow of the interaction in the Patient Interface subsystem 1 may be determined, at least in part, by information or commands from the Conversation and Session Management subsystem 2, the Medical Inference subsystem 5, the Data Management subsystem 3, or any suitable combination thereof.

The Patient Interface subsystem 1 may be realized with any one or more of a variety of available open-source libraries, third-party services, implementations customized to a particular DH-OPPT realization, the methods described herein, or any suitable combination thereof. The Patient Interface subsystem 1 may be implemented to specifically leverage the unique benefits of the DH-OPPT system, as provided by one or more of the other subsystems described herein.

The various combinations of functions described herein for the Patient Interface subsystem 1 may provide one or more benefits, such as streamlined patient interaction, integrated use of all available data, automated use of all available data, streamlined physician interactions with the patient and the information processing system (e.g., the DH-OPPT system), or any suitable combination thereof.

### 2 - Example Conversation and Session Management Subsystem

The Conversation and Session Management subsystem 2, according to various example embodiments, drives the first portion of patient interaction with the DH-OPPT system through the point of being ready for scheduling with a physician-level user (e.g., a physician or other physician-level healthcare provider). The Conversation and Session Management subsystem 2 may implement advanced-capability conversation management functionality that minimizes the number of questions asked of the patient, maximizes the medical information content of those questions, provides a natural conversational experience for the patient, or any suitable combination thereof. The Conversation and Session Management subsystem 2 achieves this improved efficiency through the use of graph-based conversation technology, which may work in conjunction with the Medical Inference subsystem 5. The Medical Inference 5 may use data provided to it by the Conversation and Session Management subsystem 2 to identify conversation tokens relevant to the graph-based conversation management algorithm. Medical data may include the raw content of the present patient conversation managed by the Conversation and Session Management subsystem 2, as well as information accessed from one or more other sources, such as EHR entries (e.g., provided by the Data Management subsystem 3). The Conversation and Session Management subsystem 2 may use data from the EHR directly, as well as in the form of derived tokens identified by the Medical Inference subsystem 5, to skip irrelevant questions and question sequences, to ask follow-up questions, or both, making for a natural conversation with the patient.

The conversation management functionality of the Conversation and Session Management subsystem 2 may be realized with any one or more of a variety of available open source libraries, third-party services (e.g., one or more bots or bot services), implementations customized to a particular DH-OPPT realization, the methods described herein, or any suitable combination thereof. Such conversation management and control may be realized in a way to achieve the summative capabilities of a DH-OPPT realization that implicitly and explicitly seeks to elicit answers to tokenized data elements used by the Medical Inference subsystem 5. The conversation management algorithms may also be driven by the Medical Inference subsystem 5, which may provide feedback, such as in the example forms of new tokens, question topics, questions, question re-phrases, or any suitable combination thereof.

The graph-based conversation technology may be implemented with any of a variety of available open-source libraries, third-party services, implementations customized to a particular DH-OPPT realization, one or more of the methods described herein, or any suitable combination thereof. Such functionality may be implemented to provide the state-based Conversation and Session Management subsystem 2 with one or more stateless data elements, one or more conversational node traversal paths, question selection and formation data, or any suitable combination thereof. Such a graph-based conversation implementation may provide such elements to, and receives such elements from, the Medical Inference subsystem 5. The Conversation and Session Management subsystem 2 may be implemented to specifically leverage the unique benefits of the DH-OPPT system, as provided by one or more of the other subsystems described herein.

The various combinations of functions described herein for the Conversation and Session Management subsystem 2 may provide one or more benefits, such as streamlined patient interaction, integrated use of all available data, automated use of all available data, maximized use of all available data, streamlined physician interactions with the patient and the information processing system (e.g., the DH-OPPT system), automated interaction events and data resolution, or any suitable combination thereof.

### 3 - Example Data Management Subsystem

The Data Management subsystem 3, according to various example embodiments, interfaces (e.g., directly) to any one or more of the other subsystems. The Data Management subsystem 3 may store all system data to be later retrieved in an access-controlled secure environment (e.g., in any one or more of the user interfaces described herein) and may provide an interface to external data by way of the External Data Gateway subsystem 11. The Data Management subsystem 3 may provide automatic PII detection and anonymization at interface boundaries across which PII transmission is not allowed. PII detection and anonymization may be achieved through any one or more of a variety of available open-source libraries, third party services, implementations customized to a particular DH-OPPT realization, the methods described herein, or any suitable combination thereof.

Each external subsystem and service may have individual access credentialing and access controls, which may limit access of that external subsystem or service to the minimum level for the subsystem or service to operate. This access credentialing and control may be realized by any one or more of a variety of available open-source libraries, third-party services, implementations customized to a particular DH-OPPT realization, the methods described herein, or any suitable combination thereof. The Data Management subsystem 3 may be implemented to specifically leverage the unique benefits of the DH-OPPT system, as provided by one or more of the other subsystems described herein.

The various combinations of functions described herein for the Data Management subsystem 3 may provide one or more benefits, such as integrated use of all available data, automated use of all available data, or any suitable combination thereof.

### 4 - Example Technician in the Loop Subsystem

The Technician in the Loop subsystem 4, according to various example embodiments, provides an interface for display, modification, and approval of information, such as by a qualified medical technician user who may supervise a patient conversation and may supervise generation of an information summary (e.g., prior to handoff of the encounter to a physician-level user). The interface of the Technician in the Loop subsystem 4 affords efficient and accurate conversation management, information labeling, and information approval by the medical technician user, who is able to handle multiple federated tasks across multiple patient conversations (e.g., simultaneously), while ensuring that health information is kept private and secure (e.g., using encryption, access control, privacy enforcement, de-identification, or any suitable combination thereof). The Technician in the Loop subsystem 4 may be driven by the Conversation and Session Management subsystem 2, with data access provided to drive the primary patient conversation, to produce the final derivative conversation produced by the Physician Data Preparation and Dynamic Scheduler subsystem 6, or both.

In the interface provided by the Technician in the Loop subsystem 4, the medical technician user, who may be supported by one or more services provided by the Medical Inference subsystem 5, is able to view patient dialog turns; select, label, modify, or approve patient intent; label or confirm medically-relevant terms and findings; select, approve, rephrase, or directly implement patient conversation dialog; select, modify, or approve summary findings; flag and service canonical conversation flow exceptions; or any suitable combination thereof. Exceptions may also be serviced by one or more medical technician users through this interface, though such servicing medical technician users may be drawn from a different pool of users, such as a pool of more medically trained personnel or personnel with more in-depth knowledge of system behavior or with more senior supervisory roles. The flexibility of the interface, along with data pre-qualification by the Medical Inference subsystem 5, may result in extreme user efficiency and accuracy compared to HIPS that lack the systems and methods discussed herein. The actions of medical technician users may also be used to modify, update, and train the Medical Inference subsystem 5, leading over time to increasingly autonomous system behavior, and making the Technician in the Loop subsystem 4 less and less critical over time to each and every conversation. In the limit, the Technician in the Loop subsystem 4 may primarily provide supervisory control and system review capability and may even become otherwise optional with respect to the primary system operation workflow.

The Technician in the Loop subsystem 4 may be realized with any one or more of a variety of available open-source libraries, third-party services, implementations customized to a particular DH-OPPT realization, the methods described herein, or any suitable combination thereof. The Technician in the Loop subsystem 4 may provide a network-distributed, federated task notification and servicing architecture, in which available medical technician users are notified of, and provided with an interface to, ongoing interactions with patients, other personnel, data, or any suitable combination thereof, as such interactions happen, in real time, offline, or both. The interface of the Technician in the Loop subsystem 4 enables medical technician users to manage one or more jobs, label and format data, determine one or more interaction modes, refer one or more jobs, request support, complete one or more jobs, or any suitable combination thereof.

In this context, the Medical Inference subsystem 5 may be implemented to perform data classification, perform state classification, recommend data tokens and data token labels, recommend question tokens and question phrases, recommend data summaries, or any suitable combination thereof, to the Technician in the Loop subsystem 4. The Medical Inference subsystem 5 may also be implemented to use data from the Technician in the Loop subsystem 4, such as data inputs and interface selections from medical technician users, patients, or both, to service the needs of, and improve performance through training for, the functions described herein for the Medical Inference subsystem 5 (e.g., in conjunction with one or more of the other subsystems, such as the Technician in the Loop subsystem 4). The Technician in the Loop subsystem 4 may be implemented to specifically leverage the unique benefits of the DH-OPPT system, as provided by one or more of the other subsystems described herein.

The various combinations of functions described herein for the Technician in the Loop subsystem 4 may provide one or more benefits, such as streamlined patient interaction, integrated use of all available data, automated use of all available data, maximized use of all available data, streamlined physician interactions with the patient and the information processing system (e.g., the DH-OPPT system), automated interaction events and data resolution, or any suitable combination thereof.

### 5 - Example Medical Inference Subsystem

The Medical Inference subsystem 5, according to various example embodiments, performs any one or more of a variety of analytic and predictive services for one or more of the other subsystems, directly or indirectly.

For the Conversation and Session Management subsystem 2, the Medical Inference subsystem 5 may perform named entity recognition (NER), relationship extraction, co-referencing, dialog token extraction, negation detection, medical condition inference, topic and question generation, inference-based data organization, or any suitable combination thereof. The Medical Inference subsystem 5 may achieve one or more of these capabilities by using a language model that starts with a medically-aware training corpus (e.g., scispaCy) used in conjunction with a normalization service (e.g., Unified Medical Language System^{®} (UMLS^{®})), and then adds to the capability and accuracy of these starting models, such as by retraining the language model based on interface selection choices (e.g., from one or more medical technician users, one or more physician-level users, one or more patients, or any suitable combination thereof), end-state session labels, direct data labeling, or any suitable combination thereof. Using such advanced capabilities to drive the patient conversation may provide one or more benefits to the patient, as well as to medical users of the system. Examples of this aspect of the system in a comprehensive context are provided below.

The NER capability of the Medical Inference subsystem 5 may perform state-of-the-art entity recognition (e.g., entity name extraction) and token recognition (e.g., token extraction), with medical context, filtering the terms according to customizable rubrics based on normalization to a standardized semantic hierarchical ontological framework. This approach may reduce clutter from terms with semantic categorization not relevant to the particular extraction task at hand, which may result in a contextually filtered result that is normalized to a standard framework. Tokens represent the lowest-level of semantic content, and as such, many derived results such as named entities are composed of tokens. Obtaining the extracted, medically relevant tokens in this way may substantially improve the ability of the DH-OPPT system to perform normalized comparisons in a machine learning framework with a finite feature space.

Building on the NER detection and filtering step just discussed, the dialog token extraction operation takes medically relevant complex-phrase detection a step further, identifying complex patterns within the input data tokens, as organized by semantic type, such as body system or pathology grouping. These complex tokens are used in large part to drive the patient interaction, identifying topics that can be skipped, as well as identifying new question topics that should be addressed. An example of the use of this component is in a typical review of systems (ROS) in the clinical medical setting; topics and body systems already covered in the preceding interview can be skipped in the ROS, enabling a much shorter and natural patient conversation without omitting any important medical information. This capability also allows for semantic and hierarchical categorization of input tokens for later use in the physician-level user interface.

Negation detection may be an important component of the capabilities of some example embodiments of the Medical Inference subsystem 5. Negation detection at the phrase level, which can be referred to more directly as “agree/disagree,” is technically challenging and not widely solved. In the Medical Inference subsystem 5, such agreement or disagreement may be detected within the patient conversation through the use of one or more of several advanced machine learning algorithms, broadly categorized as “scored” or “synthetically trained” machine learning algorithms. In the scored approach, the Medical Inference subsystem 5 use a semi-supervised lifetime learning approach to bootstrap from a small initial corpus of labeled data with an initial accuracy X, to continually and eventually learn toward a final asymptotic accuracy Y, using additional human input for a subset of new data incoming to the system. While standard deep learning models familiar to those practiced in the art may be used in the implementation, the Medical Inference subsystem 5 may differ from standard models, for example, by applying one or more of such deep learning models to arbitrarily long text passage pairs; implementing a scoring engine with a soft threshold capability that intelligently pulls out examples of the most and least ambiguous “agree/disagree” detection events in new data, such that the human supervisory role only has to deal with a very small subset of the new data, and is eventually rendered obsolete as asymptotically perfect detection accuracy is achieved; or both.

Medical condition inference is a beneficial capability, and there are two general categories of approaches to inference of medical conditions: one-off data-centric approaches and prescriptive hand-crafted approaches. In one-off data-centric approaches, the input data is featurized and used to train a machine model, often a deep neural network, to detect a single condition or infer a single continuous-valued parameter (e.g., detecting hospital revisit times or mortality dates). Such one-off data-centric approaches are just fully supervised machine learned models carefully tuned and selected for single, narrow purposes, and which depend entirely on an otherwise unexplained computation across a broad set of potentially unrelated input features that happen to be available in the data. One-off data-centric models therefore lack generalizability, lack explainability, and use large feature sets and large amounts of labeled data to be effective, the latter being unlikely to be available for all possible medical conditions. By not necessarily knowing which features are important to the detection problem ahead of time, such models must assume that all features are important, and that a large number of features must be used since the actual import of any given one is not known ahead of time.

The prescriptive hand-crafted approach essentially takes the inverse approach to the one-off data-centric approach and uses humans to carefully select from among features that are presumed, based on human understanding, to be important to a given medical condition to be detected, and which mirror what are referred to as expert systems more so than they mirror modem machine learning architectures. Prescriptive hand-crafted approaches are therefore very labor-intensive to implement for each new medical condition of interest and do not necessarily reach optimum performance, since they might not take advantage of hidden relationships in the data (e.g., between features and medical conditions), thus reducing both precision and recall.

Some example embodiments of the Medical Inference subsystem 5 apply a hybrid approach, learning from prescriptive sources (e.g., medical clinical practice guidelines (CPGs), research papers, or both) and extracting computable rules from these resources, and also learning in a data-centric way. The benefits of this approach are that the inference models originate from an explainable and acceptable source with human-parseable semantic context and meaning and contain specific derived rules as features or directly computable model nodes, while still comprising data-wholistic learning. This approach provides explainable inference as a cold-start capability, while also directly supporting one-shot and active learning in the modern sense of data-centric advanced deep learning technology. For example, the example embodiments may be capable of ingesting a free-text CPG, and, with no additional human intervention, producing an initial distributional model of the condition represented in the CPG. This distributional model may then be embedded in a deep neural network with nodes consisting of first-order logical constructs. With no additional training, most commonly implemented as “backpropagation” in the sense of modern deep neural networks, the model is able to perform inference regarding a patient’s condition, with explainability not only in the form of input features and initial weights traced back to a human-parseable CPG, but also in the form of features and their logical relations to the condition being represented as discrete nodes in the model. As labeled data is made available to the model, the model can be trained as any other deep neural network, thus improving performance while retaining a traceable and human-parseable structure to uniquely afford explainability.

Some example embodiments of the Medical Inference subsystem 5 address one of the most elusive of capabilities in science and modeling: causality. Such example embodiments may apply a model architecture that enables assessments of causality by linking causes and effects through an implicit multi-model relationship. Each medical condition model (e.g., the “effect”) may be initialized as a series of models, one for each type of cause (e.g., the “cause”). An independent model may also be initialized for each cause. The relationship between causes and effects (e.g., conditions) may be contextual, and a condition such as diabetes, for example, may be both a cause and an effect. During medical inference, the probability of each cause and effect with a relational link is evaluated, and the net condition probabilities are computed in each linked cause-effect model set. In this way, the example embodiments of the Medical Inference subsystem 5 learn to assess the existence of a cause simultaneous to the existence of an effect implicit to each cause, thus providing an overall probability of both the cause and the effect. By also instantiating additional unlabeled cause models and effect models implicitly tied to each of these causes, some example embodiments of the Medical Inference subsystem 5 can discover new causal relationships, which can later be labeled if and when such new relationships indicate higher probability than the already-labeled cause-effect pairings.

The ability of the Medical Inference subsystem 5, according to various example embodiments, to perform inference around patient medical condition, and the way in which it achieves this inference, may provide any of several benefits. For one, the Medical Inference subsystem 5 allows for driving the patient conversation in an efficient and effective way, including determination of the maximum-information topic, determination of a question that can be asked next while in-process in a conversation, or both. These capabilities enable maximization of certainty of the estimated ranking of inferred present medical conditions, as well as quantitative assessment of the state of the conversation relative to when the conversation can end without leaving potential information out. Another benefit is the ability to intelligently organize the available data around likely conditions for the physician-level user to make his or her assessments and manipulations. This intelligent organization may include the ability to represent the relative influence of individual features, traceable to resources like CPGs, to each medical condition under consideration. A benefit of this capability comes to full fruition in the Physician Encounter Interface subsystem 7, which may implement a unique physician-level user interface.

Some example embodiments of the Medical Inference subsystem 5 extend their modeling and machine learning capabilities to inference regarding both actions and outcomes. Such example embodiments may model and learn the actions most likely to be taken by the physician-level user for the patient given, not just the present medical condition inference, but also given a holistic view of the patient’s data relative to one or more globally-learned models. For example, for a given inferred medical condition, the patient’s specific EHR, demographic data, and other data, when assessed with regard to the global model, may indicate a preferred course of action of prescribing half of the average dose of a particular medication relative to the general guidance. This may provide the benefit of increasing the effectiveness of medical care by better fitting care actions to each individual, as well as enabling the discovery of new relationships between or among patient demographics, medical histories, symptoms, medical care actions, or any suitable combination thereof. As an example, suppose it is documented in CPGs that the first string drug treatment for the medical condition called “gout” is actually quite harmful to a small minority demographic group and that an alternate drug treatment should be tried instead for that group. Establishing such a relationship using conventional approaches may take many years, and there may be many such relationships to discover, in fact, with no realistic way to perform enough studies to treat each independent variable that may be in play. According to various example embodiments, the Medical Inference subsystem 5, at the very least, identifies potential factors for future study, improves performance for individual patients, and may in time be accepted as a source of bona fide proof that such relationships exist and should be taken into consideration across medical practice. This capability may lead to more accurate, individualized care of patients, as well as lower costs of healthcare by providing physician-level users with quantitative justification for skipping insurance-mandated treatment or testing steps when such treatment or testing steps are computed as likely to be ineffective.

The Medical Inference subsystem 5, according to various example embodiments, may generate derivative products (e.g., information products), such as records, patient encounter notes, care plans, or any suitable combination thereof. After a physician-level user finalizes the facts for a patient encounter, the Medical Inference subsystem 5 may organize the facts within the framework of a generative grammatical structure suitable for each type of derivative product (e.g., with final manipulation by the physician-level user through the Physician Encounter Interface subsystem 7). The Medical Inference subsystem 5 may provide one or more derivative products (e.g., as part of one or more information services) to the Note Generation subsystem 9 to specifically perform the note generation for the medical encounter, which is in turn may be used by the Billing Interface subsystem 10 to create a billing record (e.g., in compliance with ICD-10 standards).

The Medical Inference subsystem 5 may be realized with any one or more of a variety of available open-source libraries, third-party services, implementations customized to a particular DH-OPPT realization, the methods discussed herein, or any suitable combination thereof. According to various example embodiments, the Medical Inference subsystem 5 realizes machine learning (e.g., with or without one or more other analytical models) to drive conversation, assess patient condition, determine supportive actions, predict patient outcomes, generate documentation, or any suitable combination thereof. The Medical Inference subsystem 5 may have cold-start capability, lifetime learning capability, or both. Initial models may be generated using a one or more of a variety of data sources, including clinical practice guidelines, patient-physician conversations, medical articles, disease descriptions, treatment plans, EHRs, other health records, epidemiological data, other similar resources, or any suitable combination thereof. Initial models may be trained using this data to provide initial capability, and the DH-OPPT system may be updated as new data becomes available, such as patient interactions with the DH-OPPT system, physician interactions with the DH-OPPT system, patient outcomes, other updates, or any suitable combination thereof. This training action may apply one or more standard approaches, one or more customized approaches, or both, in machine learning and artificial intelligence. Such training may be performed using one or more elemental operations, such as linear regression, stochastic gradient descent, back-propagation, maximum likelihood, Bayesian techniques, or any suitable combination thereof, within one or more architectures, such as multi-layer-perceptrons, decision trees, random forests (RFs), Bayesian classifiers, convolutional neural networks, transformer networks, recurrent neural networks, cosine similarity rankings, or any suitable combination thereof. The Medical Inference subsystem 5 may be implemented to specifically leverage the unique benefits of the DH-OPPT system, as provided by one or more of the other subsystems described herein.

The various combinations of functions described herein for the Medical Inference subsystem 5 may provide one or more benefits, such as streamlined patient interaction, integrated use of all available data, automated use of all available data, maximized use of all available data, streamlined physician interactions with the patient and information processing system, automated interaction events and data resolution, or any suitable combination thereof.

### 6 - Example Physician Data Preparation and Dynamic Scheduler Subsystem

The Physician Data Preparation and Dynamic Scheduler subsystem 6, according to various example embodiments, accepts patient encounter handoffs from the Conversation and Session Management subsystem 2, and may do so while still maintaining linkages to the Technician in the Loop subsystem 4. Interoperating with the Medical Inference subsystem 5, the Data Management subsystem 3, or both, the Physician Data Preparation and Dynamic Scheduler subsystem 6 may provide primary outputs that include: a summarization of patient encounter data, a set of proposed options for case assignment and scheduling of the patient with one or more physician-level users, or both. The summarization of the patient encounter data may include data from the patient conversation, data derived from the patient conversation, data derived from one or more external resources, such as an EHR, the in-system patient record, annotations or derived products from a medical technician user (e.g., in the loop), or any suitable combination thereof. The summarization may be moderated by such a medical technician user.

The summarization of patient encounter data may be organized according to one or more rubrics with varying degrees of automated modification and organization by the Physician Data Preparation and Dynamic Scheduler subsystem 6, in some cases in conjunction with the Medical Inference subsystem 5. This automated modification and organization may facilitate maximization of the efficiency and performance of the physician-level user, and may rely on the specific interface, data formats, data manipulation capabilities, and features of the Physician Encounter Interface subsystem 7. In some example embodiments of the Physician Data Preparation and Dynamic Scheduler subsystem 6, top-level options for data organization include: organization by inferred patient conditions, and organization by problem list, with or without further top-level options.

Using the inferred patient condition option, the Physician Data Preparation and Dynamic Scheduler subsystem 6, the Medical Inference subsystem 5, or both, with optional modification by a medical technician user (e.g., via the Technician in the Loop subsystem 4), provides a list of likely diagnoses based on the available holistic patient data. The list of likely diagnoses may be provided along with one or more factors that went into the indicated potential patient conditions, one or more factors important to each condition which are not presently addressed by the current holistic patent data, or any suitable combination thereof. This may accommodate situations in which the physician-level user decides to pursue resolution or evaluation of one or more relevant but missing factors in his or her continuation of the patient encounter, if deemed relevant.

Using the problem list option, the Physician Data Preparation and Dynamic Scheduler subsystem 6, the Medical Inference subsystem 5, or both, with optional modification by a medical technician user (e.g., via the Technician in the Loop subsystem 4), provides a list of grouped symptoms and findings from the holistic patient data (e.g., according to body system, pathology type groupings, or both). Each of these groupings of symptoms and findings may be presented to the physician-level user in the Physician Encounter Interface subsystem 7 and may be resolved into one or more composite findings or assessments of condition, for example, with one or more supportive data elements indicated by the associated input symptoms or findings originally identified by the Medical Inference subsystem 5.

Whether organized by inferred conditions or by problem list, the summarization of patient encounter data may provide significantly increased efficiency and performance to the physician-level user relative to HIPS that lack the methods discussed herein. Examples of additional beneficial capabilities include automated data pre-qualification, automated data product preparation, enabling user insight and manipulation of the automated pre-populated data with advanced user interfaces, or any suitable combination thereof.

The dynamic scheduler function of the Physician Data Preparation and Dynamic Scheduler subsystem 6 automates the generation of schedule matches. Such schedule matches may be generated based on patient intent, inferred condition, medical domain of the patient encounter (e.g., as identified by the Medical Inference subsystem 5), medical domain of the physician-level user, availability of the physician-level user, privacy considerations (e.g., where there may be specific relationships between the patient and a potential physician-level user), or any suitable combination thereof. One or more of these scheduling factors may be used by the Patient Management subsystem 8 to coordinate medical care between the patient and the physician-level user. The medical care may then be interfaced respectively through the Patient Interface subsystem 1, the Physician Encounter Interface subsystem 7, or both. Once the schedule for a continuation of the patient encounter is resolved and the encounter continuation takes place, the patient and the physician-level user may interact with each other and the DH-OPPT system through any one or more of a variety of heterogeneous communications means, including text, email, chat, voice, video chat, in-person, through a software application (e.g., an app, such as a hybrid or custom software app), or any suitable combination thereof.

The Physician Data Preparation and Dynamic Scheduler subsystem 6 may be realized with any one or more of a variety of available open-source libraries, third-party services, implementations customized to a particular DH-OPPT realization, the methods described herein, or any suitable combination thereof. The Physician Data Preparation and Dynamic Scheduler subsystem 6 may be implemented to specifically leverage the unique benefits of the DH OPPT system, as provided by one or more of the other subsystems described herein.

This various combinations of functions described herein for the Physician Data Preparation and Dynamic Scheduler subsystem 6 may provide one or more benefits, such as integrated use of all available data, automated use of all available data, streamlined physician interactions with the patient and the information processing system (e.g., the DH-OPPT system), or any suitable combination thereof.

### 7 - Example Physician Encounter Interface Subsystem

The Physician Encounter Interface subsystem 7, according to various example embodiments, is provided with a rich formatted data record from the Physician Data Preparation and Dynamic Scheduler subsystem 6. Patient encounters and continuations may be coordinated through the Physician Encounter Interface subsystem 7 by the Patient Management subsystem 8, which may also coordinate one or more follow-up events once defined by the physician -level user in the Physician Encounter Interface subsystem 7. Data format reorganizations (e.g., reformulations), decision support, reference material reachback, or any suitable combination thereof, may be provided through the Physician Encounter Interface subsystem 7 by the Medical Inference subsystem 5.

In the Physician Encounter Interface subsystem 7, the physician-level user is provided with an interface that allows him or her to perform one or more of various actions, including: reviewing patient data and patient encounter data; discovering and reviewing additional patient data and patient encounter data (e.g., from resources such as the initial patient encounter conversation, derivative products of the initial holistic patient encounter, additional data in the patient history or a third-party resource, such as an EHR, an independent laboratory service, or a medication service); engaging with the patient to elicit additional information; accessing decision support materials and reference materials; adding findings, notes, other elements, or any suitable combination thereof, to the patient’s data record; arranging one or more data representations as part of the diagnostic and decision making process; evaluating one or more pre-populated options for patient treatment plans, medical action plans, or both; editing (e.g., revising, updating, modifying, or otherwise adjusting) the patient’s data record, patient treatment plan, medical action plan, or any suitable combination thereof; reviewing and manipulating one or more artifacts of the patient encounter or other relevant records, some or all of which may be pre-generated (e.g., pre-populated) by the Note Generation subsystem 9, and some others of which may be pre-generated by the Billing Interface subsystem 10; specifically defining or approving one or more follow-up items or events, as managed by the Patient Management subsystem 8; or any suitable combination thereof.

In some example embodiments, the Physician Encounter Interface subsystem 7 is able to employ one or more of a variety of physician-level interfaces (e.g., user interfaces, such a GUI) due to the rich data format provided by the Physician Data Preparation and Dynamic Scheduler subsystem 6. In some examples of the interface, the physician-level user is presented with data organized according to the inferred patient condition option or according to the problem list option, described above with respect to the Physician Data Preparation and Dynamic Scheduler subsystem 6.

Depicted in FIG. 6 is an example realization of a flexible, dynamic interface arranged according to the inferred patient condition option. In the shown interface, the physician-level user is presented with one or more inferred patient conditions (e.g., determined by the Medical Inference subsystem 5, as previously described). For each inferred condition so indicated, the Physician-level user can see the findings that support the inference of the patient condition. One or more findings that correspond to the condition and exist in the current data record may be indicated in one manner, such as by highlighting, while one or more findings that correspond to the condition but are not present in the current data record may be indicated in another manner, such as by being presented in a low-tone color. Likewise, such an interface may also indicate one or more findings that are counter-indicative of the indicated condition. The physician-level user may be enabled by the interface to manipulate one or more of the inferred patient conditions, their associated findings, or both, by one or more inputs, such as “drag and drop” or “polarity toggling,” and the physician-level user may be enabled to delete one or more conditions, delete one or more findings, instantiate one or more new conditions, instantiate one or more findings, or any suitable combination thereof. Instantiation of a condition or a finding may include its selection from a pre-generated (e.g., pre-populated) list served up by the DH-OPPT system. Such a list may be determined by the Medical Inference subsystem 5, in which the determination (e.g., pre-population) of the list may be influenced by one or more machine-learned models trained on the particular physician-level user’s past selections. Additionally, or alternatively, one or more of the machine-learned models may be trained across a larger set of users, such as within an area of practice, within a company, or across all users.

The interface shown in FIG. 6 may provide decision-support feedback, as described above. In the interface, the physician-level user is enabled to see the relative weight of each of the findings that influenced the DH-OPPT system’s selection of any one or more inferred patient conditions, including the relative weight of any conditions or findings that the user may have manually designated. Once satisfied with the selection of relevant conditions and findings, the physician-level user may edit and finalize the session record data input, and the finalized data is made available to the Data Management subsystem 3, the Patient Management subsystem 8, the Note Generation subsystem 9, or any suitable combination thereof. This interface format may be used for evaluating and selecting medical care actions after the patient’s condition has been established, for example, with support provided by the Medical Inference subsystem 5 and with one or more interactive elements of learning, as described above. The interface further may provide an assessment of expected patient outcomes, for example, based on the patient data record and selected medical care actions. This capability may be enabled by the Medical Inference subsystem 5, which may be trained across all anonymized patient records in the DH-OPPT system, thus providing the physician-level user with the ability to perform holistic data-driven simulation of the patient care landscape particular to the individual patient.

The Physician Encounter Interface subsystem 7 may provide the interface shown in FIG. 6 to the physician-level user, and the interface may provide the physician-level user with ability to review, manipulate, edit, and finalize one or more formally derived data products, such as a patient encounter note (e.g., the patient encounter note of record), a medical encounter billing record, or both. This capability may be supported by the Note Generation subsystem 9, the Billing Interface subsystem 10, or both.

The Physician Encounter Interface subsystem 7 may be realized with any one or more of a variety of available open source libraries, third-party services, implementations customized to a particular DH OPPT realization, the methods discussed herein, or any suitable combination thereof. The Physician Encounter Interface subsystem 7 may be implemented to specifically leverage the unique benefits of the DH-OPPT system, as provided by one or more of the other subsystems described herein.

The various combinations of functions described herein for the Physician Encounter Interface subsystem 7 may provide one or more benefits, such as streamlined patient interaction, integrated use of all available data, automated use of all available data, maximized use of all available data, streamlined physician interactions with the patient and information processing system, automated interaction events and data resolution, or any suitable combination thereof.

### 8 - Example Patient Management Subsystem

The Patient Management subsystem 8, according to various example embodiments, is used to track and manage patient cases within the DH-OPPT system, provide person-person communications when necessary, or both. For example, once the initial summary of the patient encounter data is completed, with or without input from (e.g., moderation by) a medical technician user (e.g., via the Technician in the Loop subsystem 4), and the physician-level user has reviewed the summarized data, then the physician-level user may decide to use the Patient Management subsystem 8 to initiate one or more patient interactions, any one or more of which may take place over a variety of media, such as text message, email, system push notification, voice, telepresence, or any suitable combination thereof. The Patient Management subsystem 8 may maintain awareness of such interactions and the state of the patient within the DH-OPPT system, for example, by tracking whether the patient has an open session that needs to be resolved, whether the patient is expected to get lab work performed, whether the patient has a follow-up appointment that needs to be scheduled, or any suitable combination thereof. The automated notifications and other interactions provided by the Patient Management subsystem 8 enable efficient case management from the standpoint of communications and recordkeeping, with select data available to the patient, system technical users, and physician-level users, each through their individual automated interfaces. In some example embodiments, the Patient Management subsystem 8 also provides the means to engage in person-to-person communications. The Patient Management subsystem 8 further may provide one or more automated interaction cues and messages (e.g., via text messaging) between or among third-party systems, such as pharmacies, laboratories, or any other healthcare related system. In certain example embodiments, however, insurance billing systems are separately handled by the Billing Interface subsystem 10.

The Patient Management subsystem 8 may be realized with any one or more of a variety of available open-source libraries, third-party services, implementations customized to a particular DH-OPPT realization, the methods described herein, or any suitable combination thereof. The Patient Management subsystem 8 may be implemented to specifically leverage the unique benefits of the DH-OPPT system, as provided by one or more of the other subsystems described herein.

The various combinations of functions described herein for the Patient Management subsystem 8 may provide one or more benefits, such as streamlined patient interaction, integrated use of all available data, automated use of all available data, streamlined physician interactions with the patient and information processing system, automated interaction events and data resolution, or any suitable combination thereof.

### 9 - Example Note Generation Subsystem

The Note Generation subsystem 9, according to various example embodiments, supports generation of derived data records (e.g., the patient encounter note of record), modification of such derived data records, approval of such derived data records, or any suitable combination thereof. The Medical Inference subsystem 5 may provide one or more services to the Note Generation subsystem 9 for generating draft versions of these derived data products, and the physician-level user, after any optional manipulations, may finalize the derived data products (e.g., using one or more of the corresponding interfaces described above, with or without one or more of the decision-support elements described above). The patient encounter note of record, in particular, may be generated using one or more of a variety of methods, including condition-specific templated methods that accrete individual natural language statements of the relevant findings and medical care actions, hybrid generative methods based on deep learning, which form typical grammatical structure as learned from a corpus of physician notes labeled by condition and actions around the patient-specific facts, or any suitable combination thereof. The latter approach takes the statistical-distributional nature of generative models, which do not guarantee any particular sequence but rather produce general grammatically-correct language sequences, and enforces injection of the patient-specific facts into the structure with 100% probability by working within a semantic framework, such as SNOMED-CT. That is, when the generative model is trained on an existing corpus of records, such as patient encounter notes of record, the patient-specific medical terms are abstracted out to a general level of semantic specificity as traversed within the semantic framework (e.g., SNOMED-CT), and those slots are then carried forward within the generative model as placeholders to be filled in for each new specific patient encounter to which the generative model will be applied. Generative statements for which the present patient data does not contain relevant findings are rejected, and following generation of the draft version of a record, the physician-level user may be given an opportunity to modify and approve the final record.

The Note Generation subsystem 9 may be realized with any one or more of a variety of available open-source libraries, third-party services, implementations customized to a particular DH-OPPT realization, the methods described herein, or any suitable combination thereof. The Note Generation subsystem 9 may be implemented using one or more of the special data resources afforded by the DH-OPPT system, which include raw data elements, as well as data elements from the Medical Inference subsystem 5, which may be selected, curated, tagged, identified, combined, derived, generated, or any suitable combination thereof. The Note Generation subsystem 9 may be implemented specifically to be manipulable by the physician-level user or other authorized user using one or more of the flexible and information-rich interfaces provided by the Physician Encounter Interface subsystem 7. The Note Generation subsystem 9 may be implemented to specifically leverage the unique benefits of a DH-OPPT system, as provided by one or more of the other subsystems described herein.

The various combinations of functions described herein for the Note Generation subsystem 9 may provide one or more benefits, such as integrated use of all available data, automated use of all available data, streamlined physician interactions with the patient and the information processing system (e.g., the DH-OPPT system), or any suitable combination thereof.

### 10 - Example Billing Interface Subsystem

The Billing Interface subsystem 10, according to various example embodiments, supports translating the patient encounter data record to one or more formats and ontologies normalized to a third-party billing interface or format, such as ICD-10.

The Billing Interface subsystem 10 may be realized with any one or more of a variety of available open-source libraries, third-party services, implementations customized to a particular DH-OPPT realization, the methods described herein, or any suitable combination thereof. The Billing Interface subsystem 10 may be implemented to provide automated billing generation for patients, third parties, or both, over a variety of media, such as electronic mail, electronic messaging, third-party applications interfaces, legacy postal systems, or any suitable combination thereof. The Billing Interface subsystem 10 may be implemented to specifically leverage the unique benefits of a DH-OPPT system, as provided by one or more of the other subsystems described herein.

The various combinations of functions described herein for the Billing Interface subsystem 10 may provide one or more benefits, such as streamlined patient interaction, integrated use of all available data, automated use of all available data, streamlined physician interactions with the patient and the information processing system (e.g., the DH-OPPT system), automated interaction events and data resolution, or any suitable combination thereof.

### 11 - External Data Gateway Subsystem

The External Data Gateway subsystem 11, according to various example embodiments, interfaces to one or more third-party systems to access or supply patient records or other data, such as reference material or system updates (e.g., code, parameter updates, or machine learning models). The External Data Gateway subsystem 11 may be implemented with high levels of security, for example, featuring automatic anonymization, encryption, identity-level service access controls, or any suitable combination thereof.

The External Data Gateway subsystem 11 may be realized with any one or more of a variety of available open-source libraries, third-party services, implementations customized to a particular DH-OPPT realization, the methods described herein, or any suitable combination thereof. The External Data Gateway subsystem 11 may be implemented to specifically leverage the unique benefits of a DH-OPPT system, as provided by one or more of the other subsystems described herein.

The various combinations of functions described herein for the External Data Gateway subsystem 11 may provide one or more benefits, such as integrated use of all available data, automated use of all available data, automated interaction events and data resolution, or any suitable combination thereof.

An example embodiment, not intended to be limiting in scope, will now be described to illuminate the collective benefits of the subsystems of the DH-OPPT system, as such subsystems combine to form a complete end-to-end DH-OPPT system that may provide several benefits to the current state of practice in the field of medicine.

In the example embodiment, a DH-OPPT system is realized with a collection of modem cloud computing resources, such as service elements from the Google Cloud^{®} computing service, implementing the functions described herein for the various subsystems described herein and realizing the capabilities of those subsystems. Such an implementation choice may afford rapid continued development and managed deployment in a way that is highly scalable and robust. In the example embodiment example, the capabilities of the DH-OPPT system are applied in a clinical medical setting.

The patient’s journey through the his or her experience with the example embodiment of the DH-OPPT system may begin with enrollment in a medical service. This enrollment may be accomplished by interfacing with the DH-OPPT system in a manner that is most convenient to the patient. Examples of suitable interfaces include: a textual chat application interface, a textual web interface, a voice interface, a video interface, in-person interaction at a physical service center, or any suitable combination thereof. Following enrollment, the patient is able to engage with the automated DH-OPPT system at any time (e.g., 24 hours a day, 7 days a week), using the interface that is most convenient for the patient.

When the patient has a medical need, the patient may initiate a new session with the automated Patient Interface subsystem 1. The Patient Interface subsystem 1 is configured to determine patient intent and respond accordingly, beginning a new encounter with a new corresponding history of present illness (HPI). The interface presented by the Patient Interface subsystem 1 allows the patient to view or modify the patient’s account details, view or modify the patient’s future scheduled interactions, view the patient’s prescribed medical care actions, access the patient’s data records, or any suitable combination thereof.

When the intent of the patient is to engage in a new encounter (e.g., with a corresponding new HPI), the automated DH-OPPT system may allocate one or more medical technician users (e.g., associated with the Technician in the Loop subsystem 4) as resources who may become potential servicers of the new encounter and who will themselves interface to the DH-OPPT system through the Technician in the Loop subsystem 4. The new encounter may be managed by the Conversation and Session Management subsystem 2 until a complete data record for the encounter (e.g., a complete encounter data record) has been obtained. The DH-OPPT system’s conversation with the patient may be driven according to a combination of a conversation management services (e.g., canonical dialog management services, intelligent conversation management services, or both), which may be afforded by a graph-based architecture that is able to fill some or all data slots with medical findings (e.g., identified using the services of the Medical Inference subsystem 5). As the patient conversation progresses, the estimates of the patient’s condition become more certain based on the conversation and other patient record data, and the DH-OPPT system may be able to ask increasingly relevant questions with regard to achieving an initial estimated differential diagnosis (DDX).

The DDX stage of the conversation may be exited after a certain number of turns of dialog has been achieved, or when the certainty metrics of the present conversation have crossed a threshold or become stable. Since the Medical Inference subsystem 5 is able to quantitatively determine the maximum-information question to ask, when this conversational process stops producing new condition probability rankings or when the change in potential re-ranking of conditions is very low, then this portion of the conversation may be deemed by the DH-OPPT system as having concluded.

Following the DDX stage of the conversation is a review of systems (ROS) stage, where the DH-OPPT system only asks about ROS elements that have not already been addressed, to keep the conversation natural and as efficient as possible. Optionally, high-criticality rule-out questions may be asked, where findings associated with potential conditions with high criticality are specifically asked about, even if such findings are not deemed highly-probable based on the encounter data obtained up to this point. This specific conversation flow is not meant to limit the present example embodiment, but rather serves to illustrate the unique flexibility and sophistication of the DH-OPPT system with regard to inference-driven patient interaction. Other conversation flows and intents may be readily supported by the graph-based conversation architecture and inference services of the example embodiment.

Within the conversation with the patient, the optional Technician in the Loop subsystem 4 may provide one or more services in the present example embodiment of the DH-OPPT system. Such services may include: conversation management and data labeling assistance, and modification and finalization of the initial data record for the encounter.

For conversation management and data labeling assistance, the Technician in the Loop subsystem 4 performs question selection, question modification, question approval, annotation of medically relevant data elements in the conversation as it progresses, or any suitable combination thereof. The Technician in the Loop subsystem 4 may flag one or more exceptions during the conversation. For example, the Technician in the Loop subsystem 4 may flag an exception if the patient intent changes midstream or if other complications with the conversation arise. When an exception is flagged, the Technician in the Loop subsystem 4 may alert one or more supervisory resources to intervene and possibly take more manual control of the patient interaction.

For the modification and finalization of the initial data record for the encounter, the Technician in the Loop subsystem 4 may review, correct, organize, or otherwise adjust, and then finalize, a summarization of the encounter data. The Technician in the Loop subsystem 4 may then pass the finalized summarization to the Physician Encounter Interface subsystem 7 (e.g., for use once a physician-level user has been scheduled to continue the encounter).

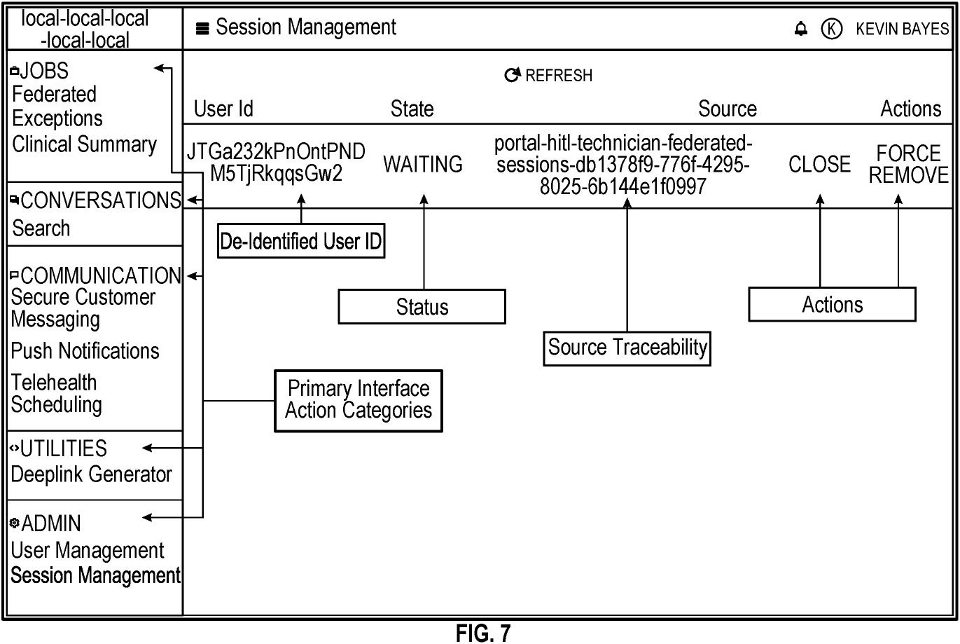

FIG. 7 is a diagram showing an example session management interface presented by the Technician in the Loop Subsystem 4 and in which one or more active sessions are listed and can be accessed, according to some example embodiments. The interface shown includes indicators of example functions performable using the interface, such as Session Access, Exception Handling, Telehealth Scheduling, Customer Messaging, and User Management, among others. The interface may be available in all modes of the interface (e.g., during performance of any of the functions of the interface).

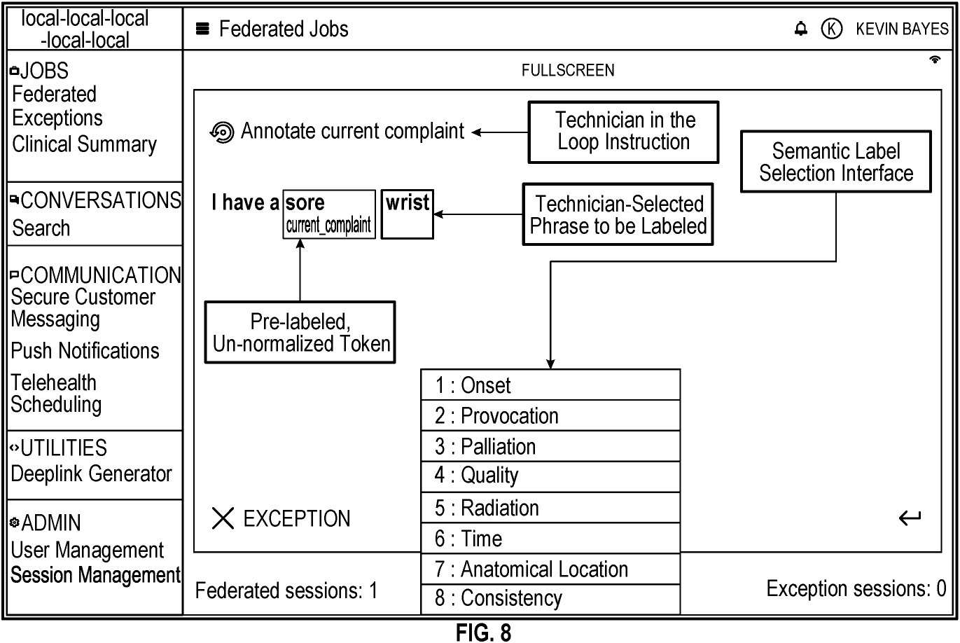

FIG. 8 is a diagram showing an example realization of an interaction between the Technician in the Loop subsystem 4 and one or more other subsystems of the DH-OPPT system, where the Technician in the Loop subsystem 4 is identifying and selecting semantically-relevant tokenized data elements, supporting or supported by one or more other subsystems, such as the Medical Inference subsystem 5 and the Conversation and Session Management subsystem 2, according to some example embodiments.

As shown in FIG. 8, the Technician in the Loop subsystem 4, during the handling of a federated session labeling event, highlights contextual tokenized information and selects from among several semantic categories to define data elements, which may be supported by one or more other subsystems, important to one or more other subsystems, or both, such as the Medical Inference subsystem 5 and the Conversation and Session Management subsystem 2.

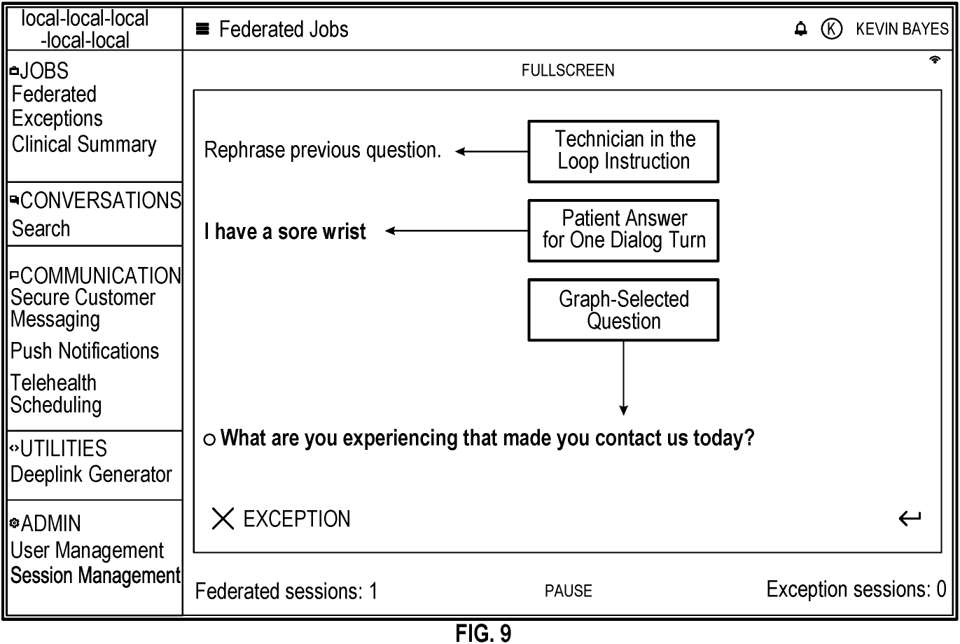

FIG. 9 is a diagram showing an example realization of an interface of the Technician in the Loop subsystem 4, where the Technician in the Loop subsystem 4 is enabled to select from various automatically derived data elements, edit such data elements, and finalize such data elements, summaries thereof, or candidate questions, according to some example embodiments. In some example embodiments, the interface provides the ability to select a system-generated data element (e.g., a fact, a summary, or a question), rephrase the selected data element, finalize the data element, or any suitable combination thereof.

One or more of the interfaces described herein (e.g., one or more GUIs) may be enabled and optimized by the summative interplay among the several subsystems of the DH-OPPT system. As a result, one or more of the interfaces described herein, including the interface shown in FIG. 9, may enable one or more benefits, including: streamlined patient interactions, integrated use of all available data, automated use of all available data, maximized use of all available data, streamlined interactions by a physician-level user with the patient and with the information processing system (e.g., the DH-OPPT system), maximized physician precision, automated interaction events and data resolution, or any suitable combination thereof.

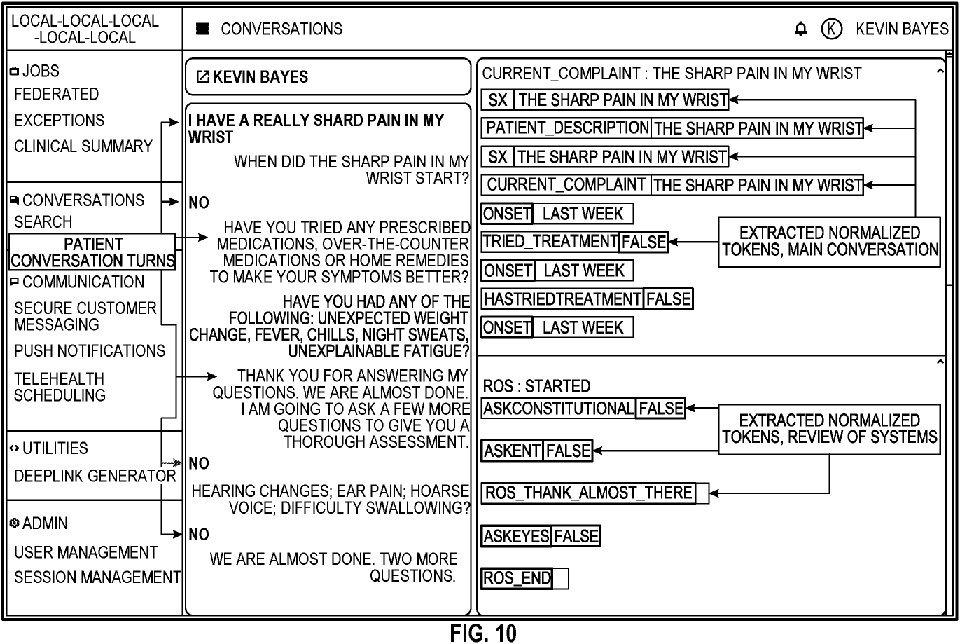

FIG. 10 is a diagram showing an example interface to a graph-based conversational element, realization, where the interface of the Technician in the Loop subsystem 4 is used to inspect the patient’s conversation with the DH-OPPT system, alongside the tokenized, state-aware, graph memory that is driving the conversation, as optionally moderated by the Technician in the Loop subsystem 4, which may work in conjunction with one or more of the other subsystems of the DH-OPPT system, according to some example embodiments. The memory of the conversational interaction between the DH-OPPT system and the patient may be implemented in a graph-based conversational structure, to obtain any one or more of the benefits described above.

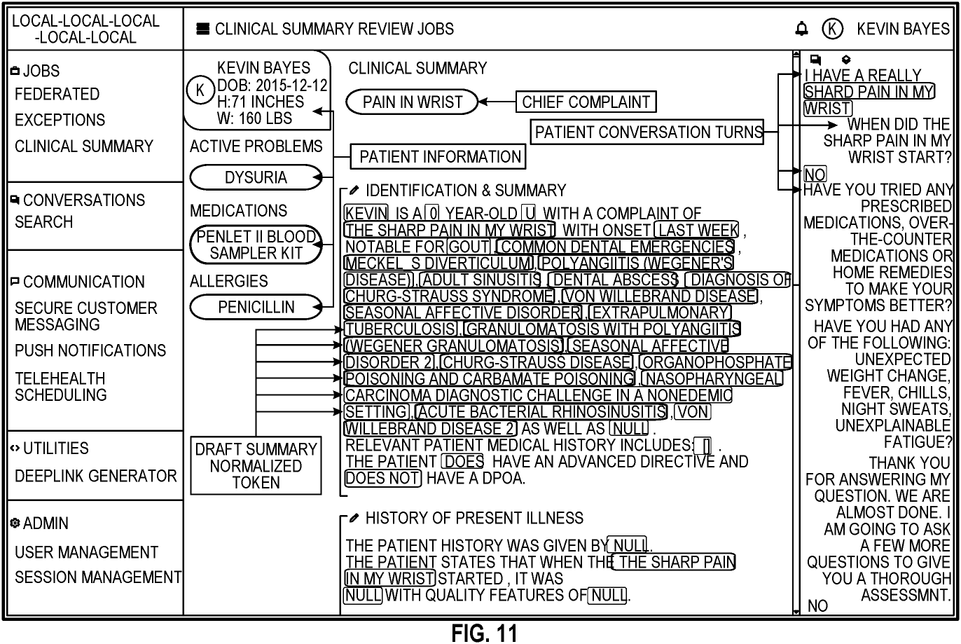

FIG. 11 is a diagram showing an example interface where the Technician in the Loop subsystem 4 is able to review, query, modify, and approve an automatically-generated and fully source-linked summary of a clinical encounter between the patient and the DH-OPPT system (e.g., before the summary is accessed by the Physician Data Preparation and Dynamic Scheduler subsystem 6), according to some example embodiments. An example of a derived data product created within the DH-OPPT system in shown in FIG. 11, in the example form of a clinical encounter summary that is automatically populated by tokenized data elements. The patient conversation on the right of the interface and the tokenized summary on the left of the interface are linked through the graph-based conversation structure and the Medical Inference subsystem 5 to provide full data traceability and explainability for both direct data elements and derived data elements anywhere along the session trajectory. In the example interface shown, the Technician in the Loop subsystem 4 is able to review, query, modify, and approve the clinical encounter summary before it is picked up by the Physician Data Preparation and Dynamic Scheduler subsystem 6.

Once the conversation and its corresponding HPI is ready for a physician-level user, the DH-OPPT system (e.g., via the Physician Data Preparation and Dynamic Scheduler subsystem 6) identifies scheduling options and communicates the scheduling options to the patient and the physician-level user in the Patient Interface subsystem 1 and the Physician Encounter Interface subsystem 7, respectively. The physician-level user may use the Physician Data Preparation and Dynamic Scheduler subsystem 6 to review the rich data record generated by the conversation, modify the record, research supporting materials, or any suitable combination thereof. The physician-level user may use the Physician Data Preparation and Dynamic Scheduler subsystem 6 to make additional scheduling choices, such as recommending an in-office visit or suggesting, for example, that a video conference sufficient for the present session with the patient would be available (e.g., by interfacing to the Patient Management subsystem 8). When an in-office continuation of a session is scheduled, the DH-OPPT system may publish this information for the benefit of one or more third parties, such as front desk staff or affiliated laboratories, through the External Data Gateway subsystem 11.

Working with the patient in a continuation of the current session, the physician-level user may perform one or more activities, such as data review, identify new data (e.g., by interacting with the patient or accessing one or more resources of the DH-OPPT system), organize findings and assessments, or any suitable combination thereof, using an interface of the Physician Encounter Interface subsystem 7. Such an interface may provide a flexible, efficient, graphically-oriented environment for such activities. Once the findings are established for the current session (e.g., and its corresponding HPI), the physician-level user may continue to use the Physician Encounter Interface subsystem 7 to assign one or more patient medical care actions, assess one or more outcomes predicted by the DH-OPPT system for the patient, or both. Such a comprehensive, data-wholistic, efficient, and predictive capability may be a welcome benefit provided by the example embodiment of the DH-OPPT system, and the example embodiment may further provide many additional indirect benefits in efficiency and quality of care. One or more follow-up actions may be automatically instantiated by the Patient Management subsystem 8, for example, including tracking one or more follow-up actions, providing one or more automated reminders (e.g., to the patient, the physician-level user, or both), scheduling one or more follow-up events, or any suitable combination thereof.

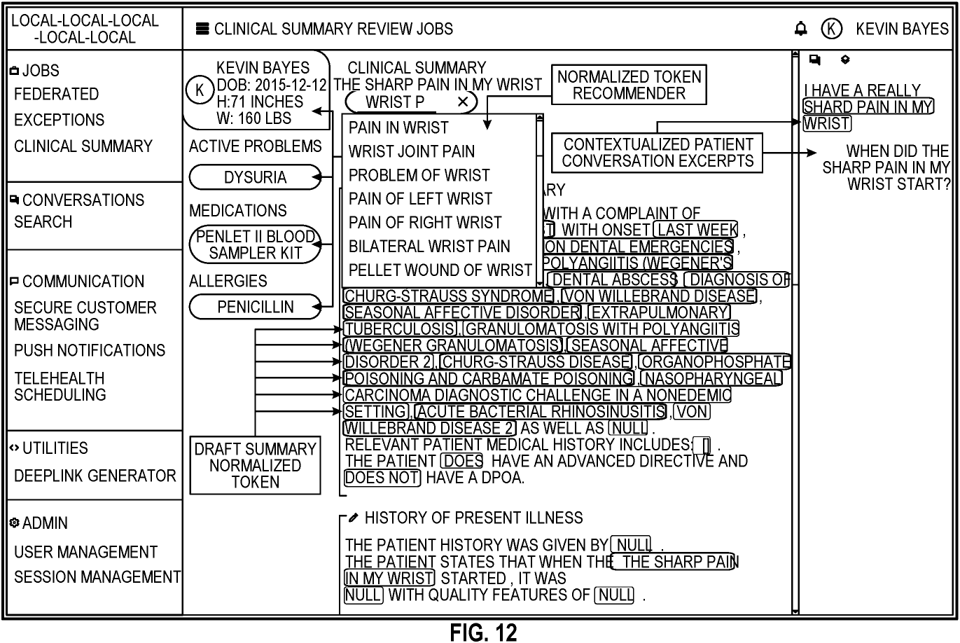

FIG. 12 is a diagram showing an example realization of an interface that enables a physician-level user to interact with a summary of a clinical encounter, where the summary features fully-traceable tokenized data and drive data elements, according to some example embodiments. Via the interface, the physician-level user is presented with an automatically-generated, optionally moderated (e.g., by a medical technician user via the Technician in the Loop subsystem 4), draft summary of the clinical encounter. The interface may be presented to the physician-level user when the physician-level user first enters the session with the patient. Any one or more of a multitude of the most relevant raw data elements and derived data products from, for example, one or more patient records, one or more patient conversations, one or more clinical guidelines, one or more medical inference sources, or any suitable combination thereof, may be presented to the physician-level user in the interface. The interface may further be a live interface (e.g., with live editing capability) that features the ability to review, query, inspect, modify, and finalize some or all of the data (e.g., with the physician-level user’s edits tracing back to some or all original source elements).

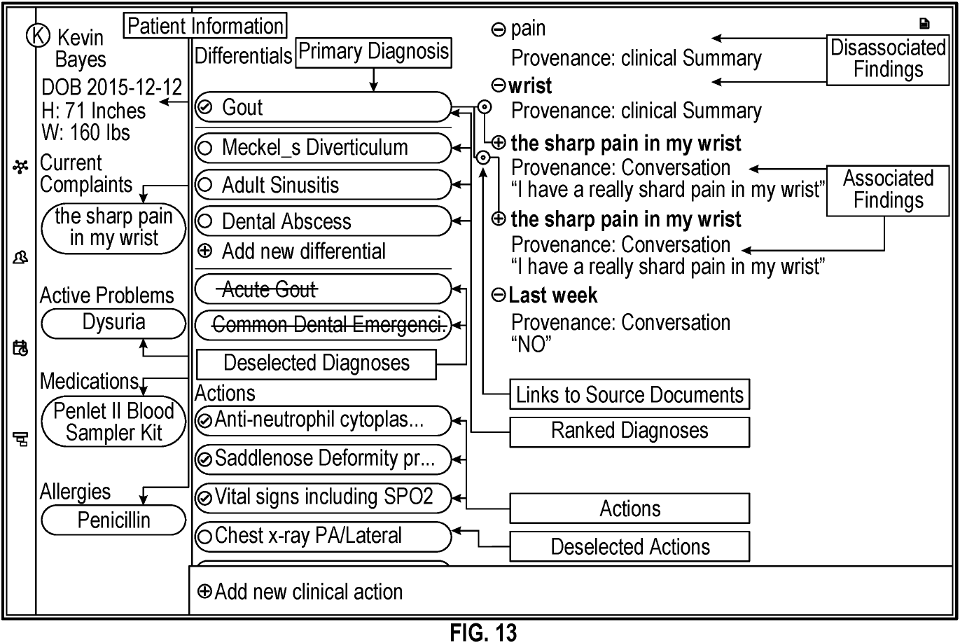

FIG. 13 is a diagram showing an example of an interface (e.g., touch-enabled) that enables a physician-level user to perform one or more diagnostic activities, with one or more displayed data elements, one or more derived data elements (e.g., derived tokens and derived objects), or both, according to some example embodiments. One or more of the derived data elements may be produced in conjunction with one or more other DH-OPPT subsystems. As with previously described interfaces, the physician-level user can use the interface to inspect any element in the interface for explainability. The interface shown in FIG. 13 may be dynamic, such that the physician-level user can move around (e.g., navigate) among various diagnoses, supporting tokens, findings, actions, data elements, derived data elements, or any suitable combination thereof, and make or break linkages between or among them relative to clinical inference. One or more new data elements can also be manually introduced by the physician-level user via the interface, as one or more outputs from the other DH-OPPT subsystems (e.g., the Medical Inference subsystem 5). The interface shown in FIG. 13 may allow for complete explainability of each data element, and each data element can be negated or re-associated.

Once the physician-level user finalizes the encounter information, which may include one or more patient actions, the record of the encounter may be made available to one or more of the other subsystems of the DH-OPPT system. The physician-level user’s net efficiency may be enhanced by the Note Generation subsystem 9, the Billing Interface subsystem 10, or both. The patient encounter note and other derivative data products may be automatically created in draft form by the Note Generation subsystem 9, the Billing Interface subsystem 10, or both, and may be presented to the physician-level user by an interface of the Patient Encounter Interface subsystem 7 (e.g., using one or more flexible, efficient, and graphically oriented environments). The patient encounter note or other derivative data products may be stored in any suitable data storage by the Data Management subsystem 3 and may be finalized or modified later by the physician-level user with full traceability. The complete DH-OPPT system, composed of a collection of the subsystems described herein, may thus provide a unique approach to the clinical medical process to achieve enhanced efficiency and accuracy at many points throughout the end to end process of providing clinical medical care.

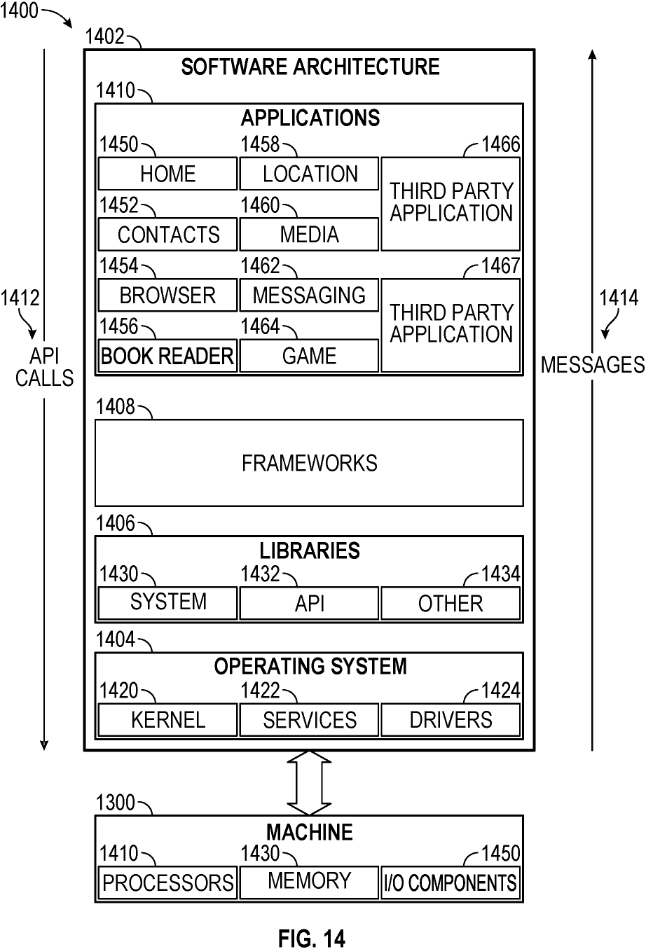

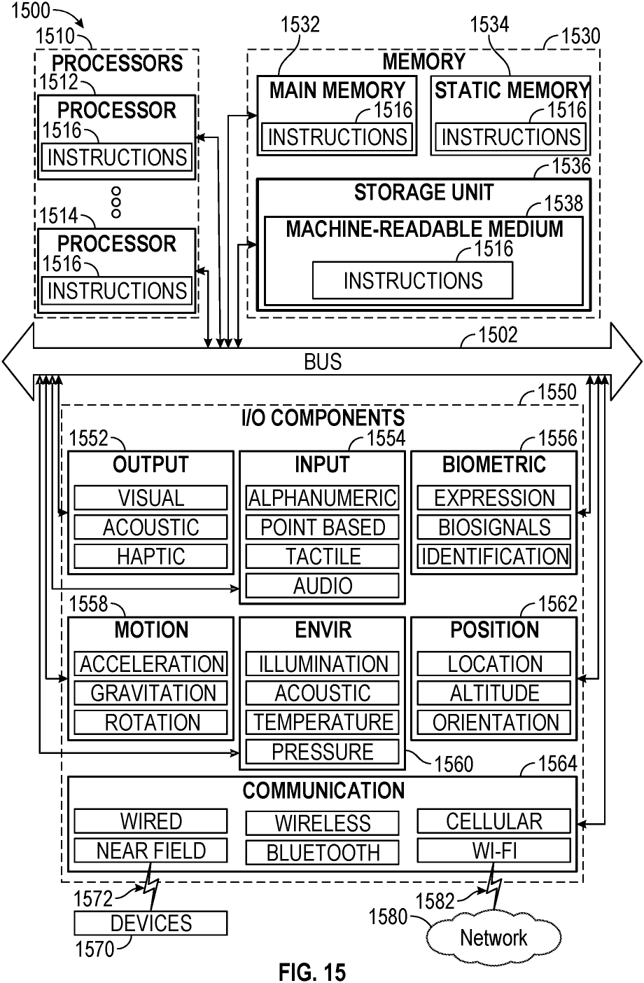

FIG. 14 is a block diagram showing an example of a software architecture for a computing device, according to some example embodiments. Specifically, a block diagram 1400 illustrates a software architecture 1402, which can be installed on any one or more of the devices described above. FIG. 14 merely illustrates a non-limiting example of the software architecture 1402, and it will be appreciated that many other architectures can be implemented to facilitate the functionality described herein. In various example embodiments, the software architecture 1402 is implemented by hardware, such as machine 1300 of FIG. 15, that includes processors 1510, memory 1530, and input/output (I/O) components 1550. In the example embodiment shown, the software architecture 1402 can be conceptualized as a stack of layers, where each layer may provide a particular functionality. For example, the software architecture 1402 includes layers, such as an operating system 1404, libraries 1406, frameworks 1408, and applications 1410. Operationally, the applications 1410 invoke application programming interface (API) calls 1412 through the software stack and receive messages 1414 in response to the API calls 1412, consistent with some example embodiments.

In various implementations, the operating system 1404 manages hardware resources and provides common services. The operating system 1404 includes, for example, a kernel 1420, services 1422, and drivers 1424. The kernel 1420 acts as an abstraction layer between the hardware and the other software layers, consistent with some example embodiments. For example, the kernel 1420 provides memory management, processor management (e.g., scheduling), component management, networking, and security settings, among other functionality. The services 1422 can provide other common services for the other software layers. The drivers 1424 are responsible for controlling or interfacing with the underlying hardware, according to some embodiments. For instance, the drivers 1424 can include display drivers, camera drivers, BLUETOOTH^{®} or BLUETOOTH^{®} Low Energy drivers, flash memory drivers, serial communication drivers (e.g., Universal Serial Bus (USB) drivers), WI-FI^{®} drivers, audio drivers, power management drivers, and so forth.

In some example embodiments, the libraries 1406 provide a low-level common infrastructure utilized by the applications 1410. The libraries 1406 can include system libraries 1430 (e.g., C standard library) that can provide functions, such as memory allocation functions, string manipulation functions, mathematical functions, and the like. In addition, the libraries 1406 can include API libraries 1432, such as media libraries (e.g., libraries to support presentation and manipulation of various media formats, such as Moving Picture Experts Group-4 (MPEG4), Advanced Video Codec (H.264 or AVC), Moving Picture Experts Group Layer-3 (MP3), Advanced Audio Coded (AAC), Adaptive Multi-Rate (AMR) audio codec, Joint Photographic Experts Group (JPEG or JPG), or Portable Network Graphics (PNG)), graphics libraries (e.g., an OpenGL framework used to render, in two dimensions (2D) or in three dimensions (3D), graphic content on a display), database libraries (e.g., SQLite to provide various relational database functions), web libraries (e.g., WebKit to provide web browsing functionality), and the like. The libraries 1406 can also include a wide variety of other libraries 1434 to provide many other APIs to the applications 1410.

The frameworks 1408 provide a high-level common infrastructure that can be utilized by the applications 1410, according to some example embodiments. For example, the frameworks 1408 provide various GUI functions, high-level resource management, high-level location services, and so forth. The frameworks 1408 can provide a broad spectrum of other APIs that can be utilized by the applications 1410, some of which may be specific to a particular operating system 1404 or platform.

In an example embodiment, the applications 1410 include a home application 1450, a contacts application 1452, a browser application 1454, a book reader application 1456, a location application 1458, a media application 1460, a messaging application 1462, a game application 1464, and a broad assortment of other applications, such as third-party applications 1466 and 1467. According to some example embodiments, the applications 1410 are programs that execute functions defined in the programs. Various programming languages can be employed to create one or more of the applications 1410, structured in a variety of manners, such as object-oriented programming languages (e.g., Objective-C, Java, or C++) or procedural programming languages (e.g., C or assembly language). In a specific example embodiment, the third-party application 1466 (e.g., an application developed using the ANDROID^{®} or IOS^{®} software development kit (SDK) by an entity other than the vendor of the particular platform) may be a mobile software app running on a mobile operating system such as IOS^{®}, ANDROID^{®}, WINDOWS^{®} Phone, or another mobile operating system. In this example embodiment, the third-party application 1466 can invoke the API calls 1412 provided by the operating system 1404 to facilitate functionality described herein.

FIG. 15 is a block diagram of a machine in the example form of a computer system, within which instructions may be executed for causing the machine to perform any one or more of the methodologies discussed herein, according to some example embodiments. Specifically, FIG. 15 illustrates components of a machine 1500 able to read instructions from a machine-readable medium (e.g., a machine readable storage medium) and perform any one or more of the methodologies discussed herein. The machine 1500 may take the example form of a computer system, within which instructions 1516 (e.g., software, a program, an application 1210, an applet, an app, or other executable code) for causing the machine 1500 to perform any one or more of the methodologies discussed herein can be executed. In alternative example embodiments, the machine 1500 operates as a standalone device or can be coupled (e.g., networked) to one or more other machines. The machine 1500 can comprise, but not be limited to, a server computer, a client computer, a personal computer (PC), a tablet computer, a laptop computer, a netbook, a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1516, sequentially or otherwise, that specify actions to be taken by the machine 1500. Further, while only a single machine 1500 is illustrated, the term “machine” shall also be taken to include a collection of machines 1500 that individually or jointly execute the instructions 1516 to perform any one or more of the methodologies discussed herein.

In various example embodiments, the machine 1500 comprises processors 1510, memory 1530, and I/O components 1550, which can be configured to communicate with each other via a bus 1502. In an example embodiment, the processors 1510 (e.g., a central processing unit (CPU), a reduced instruction set computing (RISC) processor, a complex instruction set computing (CISC) processor, a graphics processing unit (GPU), a digital signal processor (DSP), an application specific integrated circuit (ASIC), a radiofrequency integrated circuit (RFIC), another processor, or any suitable combination thereof) include, for example, a processor 1512 and a processor 1514 that may execute the instructions 1516. The term “processor” is intended to include multi-core processors 1510 that may comprise two or more independent processors 1512, 1514 (also referred to as “cores”) that can execute instructions 1516 contemporaneously. Although FIG. 15 shows multiple processors 1510, the machine 1500 may include a single processor 1510 with a single core, a single processor 1510 with multiple cores (e.g., a multi-core processor 1510), multiple processors 1512, 1514 with a single core, multiple processors 1512, 1514 with multiples cores, or any suitable combination thereof.

The memory 1530 comprises a main memory 1532, a static memory 1534, and a storage unit 1536 accessible to the processors 1510 via the bus 1502, according to some example embodiments. The storage unit 1536 can include a machine-readable medium 1538 on which are stored the instructions 1516 embodying any one or more of the methodologies or functions described herein. The instructions 1516 can also reside, completely or at least partially, within the main memory 1532, within the static memory 1534, within at least one of the processors 1510 (e.g., within the processor’s cache memory), or any suitable combination thereof, during execution thereof by the machine 1500. Accordingly, in various example embodiments, the main memory 1532, the static memory 1534, and the processors 1510 are considered machine-readable media 1538.

As used herein, the term “memory” refers to a machine-readable medium 1538 able to store data temporarily or permanently and may be taken to include, but not be limited to, random-access memory (RAM), read-only memory (ROM), buffer memory, flash memory, and cache memory. While the machine-readable medium 1538 is shown, in an example embodiment, to be a single medium, the term “machine-readable medium” should be taken to include a single medium or multiple media (e.g., a centralized or distributed database, or associated caches and servers) able to store the instructions 1516. The term “machine-readable medium” shall also be taken to include any medium, or combination of multiple media, that is capable of storing instructions (e.g., instructions 1516) for execution by a machine (e.g., machine 1500), such that the instructions 1516, when executed by one or more processors of the machine 1500 (e.g., processors 1510), cause the machine 1500 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” shall accordingly be taken to include, but not be limited to, one or more data repositories in the form of a solid state memory (e.g., flash memory), an optical medium, a magnetic medium, other non-volatile memory (e.g., erasable programmable read-only memory (EPROM)), or any suitable combination thereof. The term “machine-readable medium” specifically excludes non-statutory signals per se.

The I/O components 1550 include a wide variety of components to receive input, provide output, produce output, transmit information, exchange information, capture measurements, and so on. In general, it will be appreciated that the I/O components 1550 can include many other components that are not shown in FIG. 15. The I/O components 1550 are grouped according to functionality merely for simplifying the following discussion, and the grouping is in no way limiting. In various example embodiments, the I/O components 1550 include output components 1552 and input components 1554. The output components 1552 include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor), other signal generators, and so forth. The input components 1554 include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instruments), tactile input components (e.g., a physical button, a touch screen that provides location and force of touches or touch gestures, or other tactile input components), audio input components (e.g., a microphone), and the like.

In some example embodiments, the I/O components 1550 include biometric components 1556, motion components 1558, environmental components 1560, or position components 1562, among a wide array of other components. For example, the biometric components 1556 include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 1558 include acceleration sensor components (e.g., accelerometer), gravitation sensor components, rotation sensor components (e.g., gyroscope), and so forth. The environmental components 1560 include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensor components (e.g., machine olfaction detection sensors, gas detection sensors to detect concentrations of hazardous gases for safety or to measure pollutants in the atmosphere), or other components that may provide indications, measurements, or signals corresponding to a surrounding physical environment. The position components 1562 include location sensor components (e.g., a Global Positioning System (GPS) receiver component), altitude sensor components (e.g., altimeters or barometers that detect air pressure from which altitude may be derived), orientation sensor components (e.g., magnetometers), and the like.

Communication can be implemented using a wide variety of technologies. The I/O components 1550 may include communication components 1564 operable to couple the machine 1500 to a network 1580 or devices 1570 via a coupling 1582 and a coupling 1572, respectively. For example, the communication components 1564 include a network interface component or another suitable device to interface with the network 1580. In further examples, communication components 1564 include wired communication components, wireless communication components, cellular communication components, near-field communication (NFC) components, BLUETOOTH^{®} components (e.g., BLUETOOTH^{®} Low Energy), WI-FI^{®} components, and other communication components to provide communication via other modalities. The devices 1570 may be another machine 1500 or any of a wide variety of peripheral devices (e.g., a peripheral device coupled via a Universal Serial Bus (USB)).

Moreover, in some example embodiments, the communication components 1564 detect identifiers or include components operable to detect identifiers. For example, the communication components 1564 include radio frequency identification (RFID) tag reader components, NFC smart tag detection components, optical reader components (e.g., an optical sensor to detect one dimensional bar codes such as a Universal Product Code (UPC) bar code, multi-dimensional bar codes such as a Quick Response (QR) code, Aztec Code, Data Matrix, Dataglyph, MaxiCode, PDF417, Ultra Code, Uniform Commercial Code Reduced Space Symbology (UCC RSS)-2D bar codes, and other optical codes), acoustic detection components (e.g., microphones to identify tagged audio signals), or any suitable combination thereof. In addition, a variety of information can be derived via the communication components 1564, such as location via Internet Protocol (IP) geo-location, location via WI-FI^{®} signal triangulation, location via detecting a BLUETOOTH^{®} or NFC beacon signal that may indicate a particular location, and so forth.

In various example embodiments, one or more portions of the network 1580 can be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the public switched telephone network (PSTN), a plain old telephone service (POTS) network, a cellular telephone network, a wireless network, a WI-FI^{®}network, another type of network, or a combination of two or more such networks. For example, the network 1580 or a portion of the network 1580 may include a wireless or cellular network, and the coupling 1582 may be a Code Division Multiple Access (CDMA) connection, a Global System for Mobile communications (GSM) connection, or another type of cellular or wireless coupling. In this example embodiment, the coupling 1582 can implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

In example embodiments, the instructions 1516 are transmitted or received over the network 1580 using a transmission medium via a network interface device (e.g., a network interface component included in the communication components 1564) and utilizing any one of a number of well-known transfer protocols (e.g., Hypertext Transfer Protocol (HTTP)). Similarly, in other example embodiments, the instructions 1516 are transmitted or received using a transmission medium via the coupling 1572 (e.g., a peer-to-peer coupling) to the devices 1570. The term “transmission medium” shall be taken to include any intangible medium that is capable of storing, encoding, or carrying the instructions 1516 for execution by the machine 1500, and includes digital or analog communications signals or other intangible media to facilitate communication of such software.

Furthermore, the machine-readable medium 1538 is non-transitory (in other words, not having any transitory signals) in that it does not embody a propagating signal. However, labeling the machine-readable medium 1538 “non-transitory” should not be construed to mean that the medium is incapable of movement; the medium 1538 should be considered as being transportable from one physical location to another. Additionally, since the machine-readable medium 1538 is tangible, the medium 1538 may be considered to be a machine-readable device.

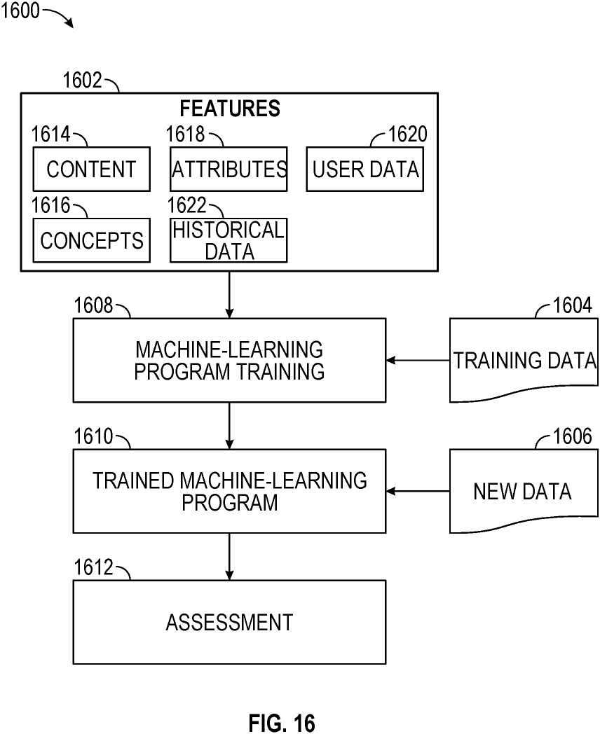

FIG. 16 is a diagram showing an example of training and use of a machine learning program 1600 that may be used to deploy various example embodiments of any one or more of the systems and methodologies discussed herein. Machine learning programs (MLPs), also referred to as machine learning algorithms or tools, are used to perform operations associated with searches, such as job searches. Machine learning is a field of study that gives computers the ability to learn without being explicitly programmed. Machine learning explores construction of algorithms, also referred to herein as tools, that may learn from existing data and make predictions about new data. Such machine learning tools operate by building a model from example training data 1604 in order to make data-driven predictions or decisions expressed as outputs or assessments (e.g., assessment 1612). Although example embodiments are presented with respect to a few machine learning tools, the principles presented herein may be applied to other machine learning tools.

In some example embodiments, different machine learning tools may be used. For example, logistic regression (LR), Naive-Bayes, RF, neural networks (NN), matrix factorization, support vector machine (SVM) tools, or any suitable combination thereof, may be used for classifying or scoring data elements or other objects (e.g., job postings).

Two common types of problems in machine learning are classification problems and regression problems. Classification problems, also referred to as categorization problems, aim at classifying items into one of several category values (e.g., is this object an apple or an orange?). Regression algorithms aim at quantifying some items (e.g., by providing a value that is a real number).

The machine learning algorithms use features 1602 for analyzing the data to generate an assessment 1612. Each of the features 1602 is an individual measurable property of a phenomenon being observed. The concept of a feature is related to that of an explanatory variable used in statistical techniques, such as linear regression. Choosing informative, discriminating, and independent features is important for the effective operation of the MLP in pattern recognition, classification, and regression. Features may be of different types, such as numeric features, strings, and graphs.

In one example embodiment, the features 1602 may be of different types and may include one or more of content 1614, concepts 1616, attributes 1618, historical data 1622, user data 1620, or any suitable combination thereof, merely for example.

The machine learning algorithms use the training data 1604 to find correlations among the identified features 1602 that affect the outcome or assessment 1612. In some example embodiments, the training data 1604 includes labeled data, which is known data for one or more identified features 1602 and one or more outcomes, such as detecting communication patterns, detecting the meaning of a message, generating a summary of the message, detecting action items in the message, detecting urgency in the message, detecting a relationship of the user to the sender, calculating score attributes, calculating message scores, etc.

With the training data 1604 and the identified features 1602, the machine learning tool is trained at machine learning program training 1608. The machine learning tool appraises the value of the features 1602 as they correlate to the training data 1604. The result of the training is the trained machine learning program 1610.

When the trained machine learning program 1610 is used to perform an assessment, new data 1606 is provided as an input to the trained machine learning program 1610, and the trained machine learning program 1610 generates the assessment 1612 as output.

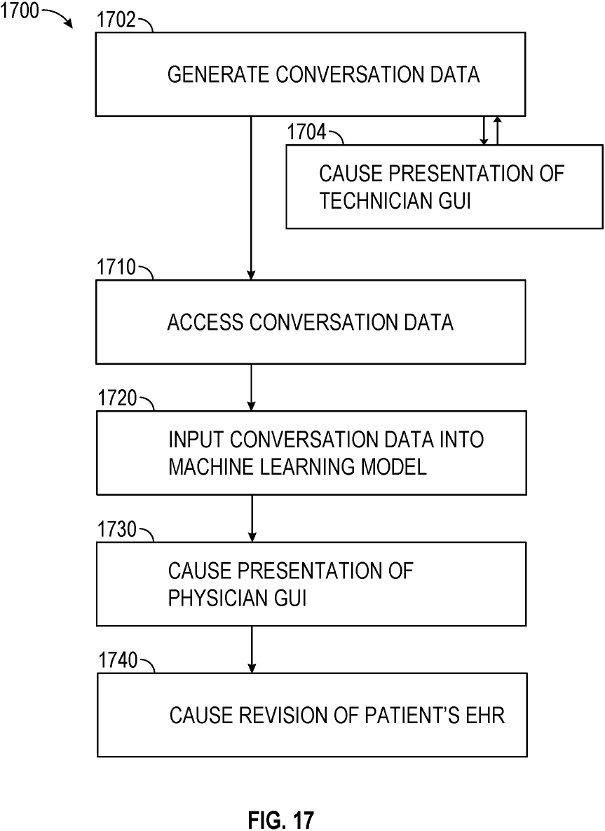

FIG. 17 is a flowchart showing a method 1700 of operating a DH-OPPT system, according to some example embodiments. Operations in the method 1700 may be performed by the DH-OPPT system, using components (e.g., subsystems or other modules) described above with respect to FIG. 1, using one or more processors (e.g., microprocessors or other hardware processors), or using any suitable combination thereof. As shown in FIG. 17, the method 1700 includes any one or more of operations 1702, 1704, 1710, 1720, 1730, and 1740.

In operation 1702, the DH-OPPT system generates one or more pairs of text passages from a dialog between the DH-OPPT system and a patient. The generation of such pairs of text passages may be performed by causing a conversation subsystem (e.g., the Patient Interface subsystem 1, the Conversation and Session Management subsystem 2, or both ) to participate in the dialog with the patient and obtain answers to questions asked of the patient. Various details of the example embodiments described above may also be incorporated in performing operation 1702.

In operation 1704, the Technician in the Loop subsystem 4 causes a corresponding GUI to present a medical technician user with at least a portion of the dialog between the patient and the conversation subsystem. As noted above, the GUI of the Technician in the Loop subsystem 4 may include a control element that is operable to finalize an answer among the obtained answers to the questions asked of the patient. In some example embodiments, the GUI of the Technician in the Loop subsystem 4 includes another control element operable to modify the answer. Various details of the example embodiments described above may also be incorporated in performing operation 1702.

In operation 1710, the Medical Inference subsystem 5 of the DH-OPPT system accesses conversation data from the encounter between the patient and the DH-OPPT system. The conversation data may include pairs of text passages from a dialog with a patient. Such pairs may include text passages of arbitrary length, as described above with respect to the Medical Inference subsystem 5. Various details of the example embodiments described above may also be incorporated in performing operation 1710.

In operation 1720, the Medical Inference subsystem 5 of the DH-OPPT system inputs the conversation data into a machine learning model trained to perform inference of medical conditions based on one or more pairs of text passages. As a result, the trained machine learning model accordingly outputs an inferred medical condition of the patient in response to the inputted conversation data. Various details of the example embodiments described above may also be incorporated in performing operation 1720.

In operation 1730, the Physician Encounter Interface subsystem 7 of the DH-OPPT system causes a GUI to present a user (e.g., a physician-level user) with a control element that is operable to edit and finalize the inferred medical condition outputted by the trained machine learning model. Various details of the example embodiments described above may also be incorporated in performing operation 1730.

In operation 1740, the Data Management subsystem 3, the External Data Gateway subsystem 11, or both, in response to operation of the control element to edit and finalize the inferred medical condition of the patient, cause revision of an electronic health record of the patient based on the finalized medical condition of the patient. Various details of the example embodiments described above may also be incorporated in performing operation 1740.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated or described. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the present subject matter has been described with reference to specific example embodiments, various modifications and changes may be made to these example embodiments without departing from the broader scope of the present disclosure.

The example embodiments discussed herein are described in sufficient detail to enable those skilled in the art to practice the teachings disclosed. Other example embodiments may be used and derived therefrom, such that structural and logical substitutions and changes may be made without departing from the scope of the present subject matter. The Detailed Description, therefore, is not to be taken in a limiting sense, and the scope of various example embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various example embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

In view of the disclosure above, various examples are set forth below. It should be noted that one or more features of an example, taken in isolation or combination, should be considered within the disclosure of this application.

A first example provides a method comprising:
accessing, by one or more processors of a machine, conversation data that includes one or more pairs of text passages from a dialog with a patient;
inputting, by the one or more processors of the machine, the conversation data into a machine learning model trained to perform inference of medical conditions based on the one or more pairs of text passages, the trained machine learning model outputting an inferred medical condition of the patient in response to the inputted conversation data;
causing, by the one or more processors of the machine, a graphical user interface to present a user with a control element operable to edit the inferred medical condition outputted by the trained machine learning model; and
causing, by the one or more processors of the machine and in response to operation of the control element to edit the inferred medical condition of the patient, revision of an electronic health record of the patient based on the edited medical condition of the patient.

A second example provides a method according to the first example, wherein:
the one or more pairs of text passages have arbitrary length;
the conversation data represents the one or more pairs of text passages of arbitrary length from the dialog with the patient; and
the machine learning model is trained to perform inference of medical conditions based on the one or more pairs of arbitrarily long text passages.

A third example provides a method according to the first example or the second example, further comprising:

generating the one or more pairs of text passages from the dialog by causing a conversation subsystem to participate in the dialog with the patient and obtain answers to questions asked of the patient.

A fourth example provides a method according to the third example, wherein:
the control element is a first control element included in the graphical user interface; and
the method further comprises:
causing a further graphical user interface to present a further user with at least a portion of the dialog between the patient and the conversation subsystem, the further graphical user interface including a second control element operable to finalize an answer among the obtained answers to the questions asked of the patient.

A fifth example provides a method according to the fourth example, wherein:

the further graphical user interface presented to the further user includes a third control element operable to edit the answer among the obtained answers to the questions asked of the patient.

A sixth example provides a method according to any of the first through third examples, wherein:
the control element is a first control element included in the graphical user interface; and
the method further comprises:
causing a further graphical user interface to present the user with a second control element operable to select whether a first list of inferred diagnoses is to be displayed in the further graphical user interface.

A seventh example provides a method according to the sixth example, wherein:

the second control element is operable to select whether the first list of inferred diagnoses or a second list of grouped symptoms is to be displayed in the further graphical user interface.

An eighth example provides a machine-readable medium (e.g., a non-transitory machine-readable medium) comprising instructions that, when executed by one or more processors of a machine, cause the machine to perform operations comprising:
accessing conversation data that includes one or more pairs of text passages from a dialog with a patient;
inputting the conversation data into a machine learning model trained to perform inference of medical conditions based on the one or more pairs of text passages, the trained machine learning model outputting an inferred medical condition of the patient in response to the inputted conversation data;
causing a graphical user interface to present a user with a control element operable to edit the inferred medical condition outputted by the trained machine learning model; and
causing, in response to operation of the control element to edit the inferred medical condition of the patient, revision of an electronic health record of the patient based on the edited medical condition of the patient.

A ninth example provides a machine-readable medium according to the eighth example, wherein:
the one or more pairs of text passages have arbitrary length;
the conversation data represents the one or more pairs of text passages of arbitrary length from the dialog with the patient; and
the machine learning model is trained to perform inference of medical conditions based the one or more pairs of arbitrarily long text passages.

A tenth example provides a machine-readable medium according to the eighth example or the ninth example, wherein the operations further comprise:

generating the one or more pairs of text passages from the dialog by causing a conversation subsystem to participate in the dialog with the patient and obtain answers to questions asked of the patient.

An eleventh example provides a machine-readable medium according to the tenth example, wherein:
the control element is a first control element included in the graphical user interface; and
the operations further comprise:
causing a further graphical user interface to present a further user with at least a portion of the dialog between the patient and the conversation subsystem, the further graphical user interface including a second control element operable to finalize an answer among the obtained answers to the questions asked of the patient.

A twelfth example provides a machine-readable medium according to the eleventh example, wherein:

the further graphical user interface presented to the further user includes a third control element operable to edit the answer among the obtained answers to the questions asked of the patient.

A thirteenth example provides a machine-readable medium of according to any of the eighth through tenth examples, wherein:
the control element is a first control element included in the graphical user interface; and
the operations further comprise:
causing a further graphical user interface to present the user with a second control element operable to select whether a first list of inferred diagnoses is to be displayed in the further graphical user interface.

A fourteenth example provides a machine-readable medium according to the thirteenth example, wherein:

the second control element is operable to select whether the first list of inferred diagnoses or a second list of grouped symptoms is to be displayed in the further graphical user interface.

A fifteenth example provides a system (e.g., a DH-OPPT system, computer system, or other system of one or more machines) comprising:
one or more processors; and
a memory storing instructions that, when executed by at least one processor among the one or more processors, cause the system to perform operations comprising:
accessing conversation data that includes one or more pairs of text passages from a dialog with a patient;
inputting the conversation data into a machine learning model trained to perform inference of medical conditions based on the one or more pairs of text passages, the trained machine learning model outputting an inferred medical condition of the patient in response to the inputted conversation data;
causing a graphical user interface to present a user with a control element operable to edit the inferred medical condition outputted by the trained machine learning model; and
causing, in response to operation of the control element to edit the inferred medical condition of the patient, revision of an electronic health record of the patient based on the edited medical condition of the patient.

A sixteenth example provides a system according to the fifteenth example, wherein:
the one or more pairs of text passages have arbitrary length;
the conversation data represents the one or more pairs of text passages of arbitrary length from the dialog with the patient; and
the machine learning model is trained to perform inference of medical conditions based the one or more pairs of arbitrarily long text passages.

A seventeenth example provides a system according to the fifteenth example or the sixteenth example, wherein:

generating the one or more pairs of text passages from the dialog by causing a conversation subsystem to participate in the dialog with the patient and obtain answers to questions asked of the patient.

An eighteenth example provides a system according to the seventeenth example, wherein:
the control element is a first control element included in the graphical user interface; and
the operations further comprise:
causing a further graphical user interface to present a further user with at least a portion of the dialog between the patient and the conversation subsystem, the further graphical user interface including a second control element operable to finalize an answer among the obtained answers to the questions asked of the patient.

A nineteenth example provides a system according to the eighteenth example, wherein:

the further graphical user interface presented to the further user includes a third control element operable to edit the answer among the obtained answers to the questions asked of the patient.

A twentieth example provides a system according to any of the fifteenth through seventeenth examples, wherein:
the control element is a first control element included in the graphical user interface; and
the operations further comprise:
causing a further graphical user interface to present the user with a second control element operable to select whether a first list of inferred diagnoses is to be displayed in the further graphical user interface.

A twenty-first example provides a carrier medium carrying machine-readable instructions for controlling a machine to carry out the operations (e.g., method operations) performed in any one of the previously described examples.

What is claimed is:

1. A method comprising:
accessing, by one or more processors of a machine, conversation data that includes one or more pairs of text passages from a dialog with a patient;
inputting, by the one or more processors of the machine, the conversation data into a machine learning model trained to perform inference of medical conditions based on the one or more pairs of text passages, the trained machine learning model outputting an inferred medical condition of the patient in response to the inputted conversation data;
causing, by the one or more processors of the machine, a graphical user interface to present a user with a control element operable to edit the inferred medical condition outputted by the trained machine learning model; and
causing, by the one or more processors of the machine and in response to operation of the control element to edit the inferred medical condition of the patient, revision of an electronic health record of the patient based on the edited medical condition of the patient.

2. The method of claim 1, wherein:
the one or more pairs of text passages have arbitrary length;
the conversation data represents the one or more pairs of text passages of arbitrary length from the dialog with the patient; and
the machine learning model is trained to perform inference of medical conditions based on the one or more pairs of arbitrarily long text passages.

3. The method of claim 1, further comprising:
generating the one or more pairs of text passages from the dialog by causing a conversation subsystem to participate in the dialog with the patient and obtain answers to questions asked of the patient.

4. The method of claim 3, wherein:
the control element is a first control element included in the graphical user interface; and
the method further comprises:
causing a further graphical user interface to present a further user with at least a portion of the dialog between the patient and the conversation subsystem, the further graphical user interface including a second control element operable to finalize an answer among the obtained answers to the questions asked of the patient.

5. The method of claim 4, wherein:
the further graphical user interface presented to the further user includes a third control element operable to edit the answer among the obtained answers to the questions asked of the patient.

6. The method of claim 1, wherein:
the control element is a first control element included in the graphical user interface; and
the method further comprises:
causing a further graphical user interface to present the user with a second control element operable to select whether a first list of inferred diagnoses is to be displayed in the further graphical user interface.

7. The method of claim 6, wherein:
the second control element is operable to select whether the first list of inferred diagnoses or a second list of grouped symptoms is to be displayed in the further graphical user interface.

8. A machine-readable medium comprising instructions that, when executed by one or more processors of a machine, cause the machine to perform operations comprising:
accessing conversation data that includes one or more pairs of text passages from a dialog with a patient;
inputting the conversation data into a machine learning model trained to perform inference of medical conditions based on the one or more pairs of text passages, the trained machine learning model outputting an inferred medical condition of the patient in response to the inputted conversation data;
causing a graphical user interface to present a user with a control element operable to edit the inferred medical condition outputted by the trained machine learning model; and
causing, in response to operation of the control element to edit the inferred medical condition of the patient, revision of an electronic health record of the patient based on the edited medical condition of the patient.

9. The machine-readable medium of claim 8, wherein:
the one or more pairs of text passages have arbitrary length;
the conversation data represents the one or more pairs of text passages of arbitrary length from the dialog with the patient; and
the machine learning model is trained to perform inference of medical conditions based the one or more pairs of arbitrarily long text passages.

10. The machine-readable medium of claim 8, wherein the operations further comprise:
generating the one or more pairs of text passages from the dialog by causing a conversation subsystem to participate in the dialog with the patient and obtain answers to questions asked of the patient.

11. The machine-readable medium of claim 10, wherein:
the control element is a first control element included in the graphical user interface; and
the operations further comprise:
causing a further graphical user interface to present a further user with at least a portion of the dialog between the patient and the conversation subsystem, the further graphical user interface including a second control element operable to finalize an answer among the obtained answers to the questions asked of the patient.

12. The machine-readable medium of claim 11, wherein:
the further graphical user interface presented to the further user includes a third control element operable to edit the answer among the obtained answers to the questions asked of the patient.

13. The machine-readable medium of claim 8, wherein:
the control element is a first control element included in the graphical user interface; and
the operations further comprise:
causing a further graphical user interface to present the user with a second control element operable to select whether a first list of inferred diagnoses is to be displayed in the further graphical user interface.

14. The machine-readable medium of claim 13, wherein:
the second control element is operable to select whether the first list of inferred diagnoses or a second list of grouped symptoms is to be displayed in the further graphical user interface.

15. A system comprising:
one or more processors; and
a memory storing instructions that, when executed by at least one processor among the one or more processors, cause the system to perform operations comprising:
accessing conversation data that includes one or more pairs of text passages from a dialog with a patient;
inputting the conversation data into a machine learning model trained to perform inference of medical conditions based on the one or more pairs of text passages, the trained machine learning model outputting an inferred medical condition of the patient in response to the inputted conversation data;
causing a graphical user interface to present a user with a control element operable to edit the inferred medical condition outputted by the trained machine learning model; and
causing, in response to operation of the control element to edit the inferred medical condition of the patient, revision of an electronic health record of the patient based on the edited medical condition of the patient.

16. The system of claim 15, wherein:
the one or more pairs of text passages have arbitrary length;
the conversation data represents the one or more pairs of text passages of arbitrary length from the dialog with the patient; and
the machine learning model is trained to perform inference of medical conditions based the one or more pairs of arbitrarily long text passages.

17. The system of claim 15, wherein:
generating the one or more pairs of text passages from the dialog by causing a conversation subsystem to participate in the dialog with the patient and obtain answers to questions asked of the patient.

18. The system of claim 17, wherein:
the control element is a first control element included in the graphical user interface; and
the operations further comprise:
causing a further graphical user interface to present a further user with at least a portion of the dialog between the patient and the conversation subsystem, the further graphical user interface including a second control element operable to finalize an answer among the obtained answers to the questions asked of the patient.

19. The system of claim 18, wherein:
the further graphical user interface presented to the further user includes a third control element operable to edit the answer among the obtained answers to the questions asked of the patient.

20. The system of claim 15, wherein:
the control element is a first control element included in the graphical user interface; and
the operations further comprise:
causing a further graphical user interface to present the user with a second control element operable to select whether a first list of inferred diagnoses is to be displayed in the further graphical user interface.
